(19) United States
(12) Patent Application Publication
(10) Pub. No.: US 2022/0202319 A1
(43) Pub. Date: Jun. 30, 2022

(54) MEAL AND ACTIVITY LOGGING WITH A GLUCOSE MONITORING INTERFACE

(71) Applicant: DexCom, Inc., San Diego, CA (US)

(72) Inventors: Margaret A. Crawford, Encinitas, CA (US); Linda Schertzer, San Diego, CA (US); Andrea J. Jackson, Solana Beach, CA (US); Douglas Scott Kanter, San Diego, CA (US); Giada Acciaroli, Ceggia (IT); Chad Patterson, San Diego, CA (US); Apurv Kamath, San Diego, CA (US); Alexander Michael Diener, San Diego, CA (US); Drew Terry, San Diego, CA (US); Mark Derdzinski, La Jolla, CA (US); Sarah Kate Pickus, San Diego, CA (US); Lauren Hruby Jepson, San Diego, CA (US); Adam Noar, San Diego, CA (US)

(73) Assignee: DexCom, Inc., San Diego, CA (US)

(21) Appl. No.: 17/556,822

(22) Filed: Dec. 20, 2021

Related U.S. Application Data
(60) Provisional application No. 63/131,680, filed on Dec. 29, 2020.

(51) Int. Cl. A61B 5/145; A61B 5/00; A61B 5/11

(52) U.S. Cl. CPC A61B 5/14532; A61B 5/743; A61B 5/0002; A61B 5/6801; A61B 5/1118

(57) ABSTRACT

Meal and activity logging with a glucose monitoring interface is described. A glucose monitoring application is configured to display a user interface that includes a glucose graph that plots glucose measurements of a user over time. The glucose measurements, for example, may be obtained from a glucose monitoring device that collects glucose measurements of the user at predetermined intervals, e.g., every five minutes. Unlike conventional event logging approaches, the glucose monitoring application displays representations of logged events in the user interface along with the glucose graph. The logged events, for example, may include meals consumed by the user, and/or various activities performed by the user, such as exercise, meditation, sleep, and so forth. Notably, the glucose monitoring application controls the display of the event representations to be presented at positions on the glucose graph that correspond to times associated with the respective events.

### RELATED APPLICATION

This application claims priority under 35 U.S.C. § 119(e) to U.S. Provisional Patent Application No. 63/131,680, filed Dec. 29, 2020 and titled “Meal and Activity Logging With a Glucose Monitoring Interface,” the entire disclosure of which is hereby incorporated by reference.

### BACKGROUND

Diabetes is a metabolic condition affecting hundreds of millions of people, and is one of the leading causes of death worldwide. For people living with Type I diabetes, access to treatment is critical to their survival and it can reduce adverse outcomes among people with Type II diabetes. With proper treatment, serious damage to the heart, blood vessels, eyes, kidneys, and nerves, due to diabetes can be avoided. Regardless of a type of diabetes (e.g., Type I or Type II), managing it successfully involves monitoring and oftentimes adjusting food and activity to control a person's blood glucose, such as to reduce severe fluctuations in and/or generally lower the person's glucose. Lifestyle event logging (e.g., food and activity logging) is a way to monitor the foods that a person eats and activities in which the person engages.

However, conventional approaches for logging meals and activity can be time-consuming and arduous. With meal logging, for example, conventional approaches can require a person, every time she or he eats, to identify the foods eaten and enter them into a log. Such entry can involve writing down the different foods in a physical log or typing them for entry into an electronic log. In some cases, users must also weigh or measure food in order to calculate the amount of calories, carbohydrates, protein, and fat in the meal. Many people find this practice difficult to sustain though. Moreover, while activity logging has become more commonplace and less tedious with the proliferation of mobile and wearable technology, e.g., smart phones and smart watches, the information that conventional systems display for logged activities does not indicate how such activities impact a person's glucose.

### SUMMARY

To overcome these problems, meal and activity logging with a glucose monitoring interface is leveraged. A glucose monitoring application is configured to display a user interface that includes a glucose graph that plots glucose measurements of a user over time. The glucose measurements, for example, may be obtained from a glucose monitoring device that collects glucose measurements of the user at predetermined intervals, e.g., every five minutes. Unlike conventional event logging approaches, the glucose monitoring application displays representations of logged events in the user interface along with the glucose graph. The logged events, for example, may include meals consumed by the user, and/or various activities performed by the user, such as exercise, meditation, sleep, and so forth. Notably, the glucose monitoring application controls the display of the event representations to be presented at positions on the glucose graph that correspond to times associated with the respective events.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an exemplary implementation that is operable to employ techniques described herein.

FIG. 2 depicts an example of the wearable glucose monitoring device of FIG. 1 in greater detail.

FIG. 3 depicts an example of an implementation of a user interface displaying meal logging visual representations with glucose measurements.

FIG. 4 depicts an example of an implementation of a user interface displaying activity logging visual representations with glucose measurements.

FIG. 5 depicts an example of an implementation of a user interface displaying event logging visual representations with glucose measurements.

FIG. 6 depicts an example of an implementation of a user interface for capturing an image of a meal to use with a logged meal.

FIG. 7 depicts an example of an implementation of a user interface which includes graphical elements that are user selectable to provide meal details for logging a meal.

FIG. 8 depicts an example of an implementation of a user interface displaying meal logging visual representations as text information with glucose measurements.

FIG. 9 depicts further examples of implementations of user interfaces displaying meal logging visual representations as text information with glucose measurements.

FIG. 10 depicts further examples of implementations of user interfaces displaying meal logging visual representations as meal images or icons with glucose measurements.

FIG. 11 depicts an example of an implementation of a user interface which includes graphical elements that are user selectable to provide activity details for logging an activity.

FIG. 12 depicts further examples of implementations of user interfaces displaying activity logging visual representations as activity images or icons with glucose measurements.

FIG. 13 depicts an example of an implementation of user interfaces used in connection with selecting a meal to view its historical effects on glucose measurements.

FIG. 14 depicts a further example of an implementation of user interfaces used in connection with selecting a meal to view its historical effects on glucose measurements, including historical effects on glucose measurements of a population of users.

FIG. 15 depicts another example of a user interface displaying event logging visual representations with glucose measurements.

FIG. 16 depicts a procedure in an example of an implementation in which an event representation of an event is displayed in a user interface with a glucose graph.

FIG. 17 depicts a procedure in an example of an implementation in which an event is logged responsive to user input and an event representation of the event is displayed in a user interface with a glucose graph.

FIG. 18 depicts an additional procedure in an example of an implementation in which an event representation of an event is displayed in a user interface with a glucose graph.

FIG. 19 illustrates an example of a system including various components of an example device that can be implemented as any type of computing device as described and/or utilized with reference to FIGS. 1-18 to implement embodiments of the techniques described herein.

### DETAILED DESCRIPTION

Overview

With conventional logging approaches, the meals and activities logged are divorced from how they affect a person's glucose, e.g., logged meals and activities are visually separated from the person's glucose information depicted in a glucose graph. Consequently, these techniques fail to provide enough immediate relevance to motivate a person to continue logging his or her meals and activities.

To overcome these problems, meal and activity logging with a glucose monitoring interface is leveraged. A glucose monitoring application is configured to display a user interface that includes a glucose graph that plots glucose measurements of a user over time. The glucose measurements, for example, may be obtained from a glucose monitoring device that collects glucose measurements of the user at predetermined intervals, e.g., every five minutes. The user interface may include selectable elements that are selectable to display the glucose measurements plotted over different time periods, such as a 3-hour time period, a 5-hour time period, a 12-hour time period, and a 24-hour time period.

Unlike conventional event logging approaches, the glucose monitoring application displays representations of logged events in the user interface along with the glucose graph. The logged events, for example, may include meals consumed by the user, and/or various activities performed by the user, such as exercise, meditation, sleep, and so forth. Notably, the glucose monitoring application controls the display of the event representations to be presented at positions on the glucose graph that correspond to times associated with the respective events. For example, a representation of a meal can be presented on the glucose graph at a position that corresponds to a time at which the meal was consumed by the user, while a representation of an exercise performed by the user can be presented on the glucose graph at a position that corresponds to a time at which the exercise was performed.

Presenting the event representations in conjunction with the glucose graph provides a visual correlation between an event and the impact of the event on the glucose levels of the user over the course of the subsequent minutes or hours. This visual association between events and glucose measurements can educate a user about which events help the user meet his or her health goals (e.g., help keep the user's glucose measurements within a target range) and which events may be preventing the user from meeting his or her health goals (e.g., cause the user's glucose measurements to be outside the target range). Doing so enables users to connect the dots between events and their glucose levels, and thus leads the user to make meaningful every day changes for their long-term health, such as by eating different meals, exercising more frequently, “fine-tuning” the timing of their food intake and/or physical activity, and so forth. For example, the display of a representation of white rice on a glucose graph depicting a subsequent spike in glucose values caused by consumption of the white rice may motivate the user to eat white rice less frequently. As another example, the display of an activity representation of a walk (that occurs after eating the bowl of white rice) on a glucose graph depicting that the user's glucose stayed within the target range may help educate the user that the timing of activities related to food consumption can help to control the user's glucose.

In addition to displaying the event representations along with the glucose graph, the glucose monitoring application provides an intuitive and efficient interface that enables users to quickly and easily log events, such as by logging a meal by snapping a photo of the meal or logging an exercise via a voice command. Responsive to a user input to log the event, the glucose monitoring application automatically logs the event by storing the event (e.g., a photo of a meal) along with a time associated with the event. Notably, the glucose monitoring application can determine the time associated with the logged events and automatically associate the event with the glucose graph without further input from the user. For example, a time associated with a meal may be determined by identifying a time (e.g., via a timestamp) when a photo of the meal is captured, e.g., using a camera, or when the user provides other input to log the meal, such as via text entry, dropdown box, or a voice command. Subsequently, the glucose monitoring application displays representations of the logged events with the glucose graph without any additional input from the user.

Notably, the glucose monitoring interface described herein enables the user to quickly log events while still being able to understand the impact of these events on their glucose values. For example, rather than weighing food in order to calculate and log the amount of carbohydrates in a bowl of white rice, the user can simply snap a photo of the bowl of white rice. By then displaying the photo of white rice proximate the glucose graph, the user is able to understand the impact that consumption of the white rice had on the user's glucose values. Doing so may thus provide the user with a memory of this impact such that the next time the user wants to eat white rice the user can recall the impact, or search to find the previous glucose graph in order to understand the impact that this particular food has had on the user's glucose values in the past. Moreover, by eliminating tedious logging requirements, such as weighing food to count carbohydrates or typing in each ingredient in a meal, the user is more likely to continue logging events instead of giving up in frustration. By providing users with a memory of the impact of meals and/or activities on glucose, the described techniques incentivizes users to log their meals and activities thereby improving the consistency of event logging which results in long term health benefits for the user.

In the following discussion, an exemplary environment is first described that may employ the techniques described herein. Examples of implementation details and procedures are then described which may be performed in the exemplary environment as well as other environments. Performance of the exemplary procedures is not limited to the exemplary environment and the exemplary environment is not limited to performance of the exemplary procedures.

Example of an Environment

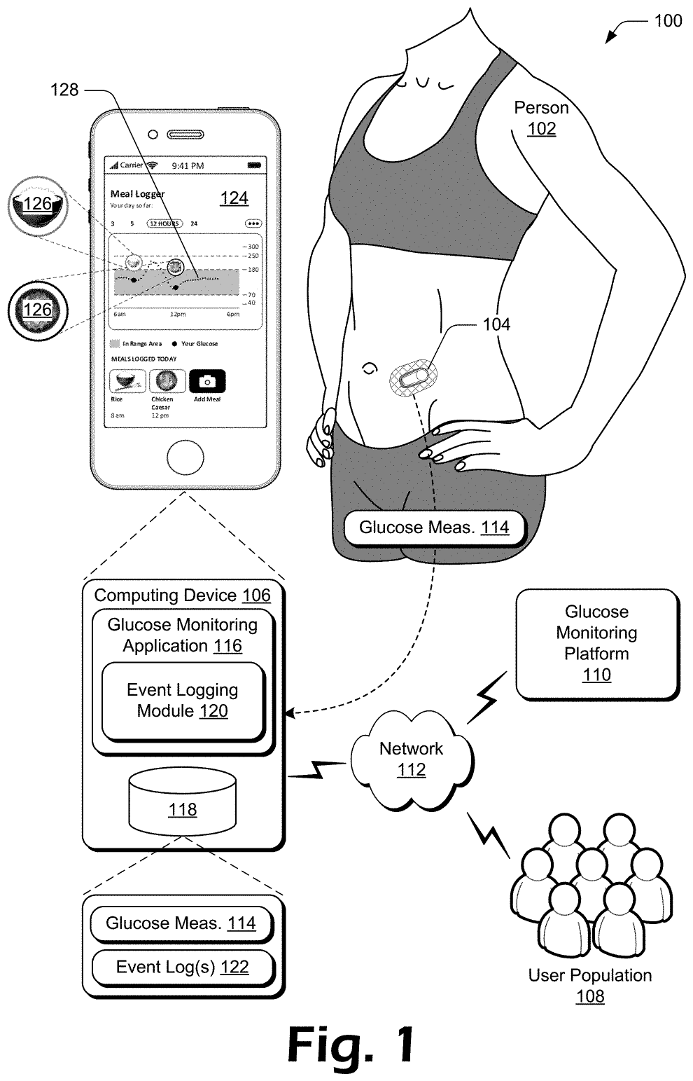

FIG. 1 is an illustration of an environment 100 in an example of an implementation that is operable to employ meal and activity logging with a glucose monitoring interface as described herein. The illustrated environment 100 includes person 102, who is depicted wearing a wearable glucose monitoring device 104. The illustrated environment 100 also includes computing device 106, other users in a user population 108 that wear glucose monitoring devices 104, and glucose monitoring platform 110. The wearable glucose monitoring device 104, computing device 106, user population 108, and glucose monitoring platform 110 are communicatively coupled, including via a network 112.

Alternately or additionally, the wearable glucose monitoring device 104 and the computing device 106 may be communicatively coupled in other ways, such as using one or more wireless communication protocols or techniques. By way of example, the wearable glucose monitoring device 104 and the computing device 106 may communicate with one another using one or more of Bluetooth (e.g., Bluetooth Low Energy links), near-field communication (NFC), 5G, and so forth.

In accordance with the described techniques, the wearable glucose monitoring device 104 is configured to provide measurements of person 102's glucose. Although a wearable glucose monitoring device is discussed herein, it is to be appreciated that meal and activity logging with a glucose monitoring user interface may be performed in connection with other devices capable of providing glucose measurements, e.g., non-wearable glucose devices such as blood glucose meters requiring finger sticks, patches, and so forth. In implementations that involve the wearable glucose monitoring device 104, though, it may be configured with a glucose sensor that continuously detects analytes indicative of the person 102's glucose and enables generation of glucose measurements. In the illustrated environment 100 and throughout the detailed description these measurements are represented as glucose measurements 114.

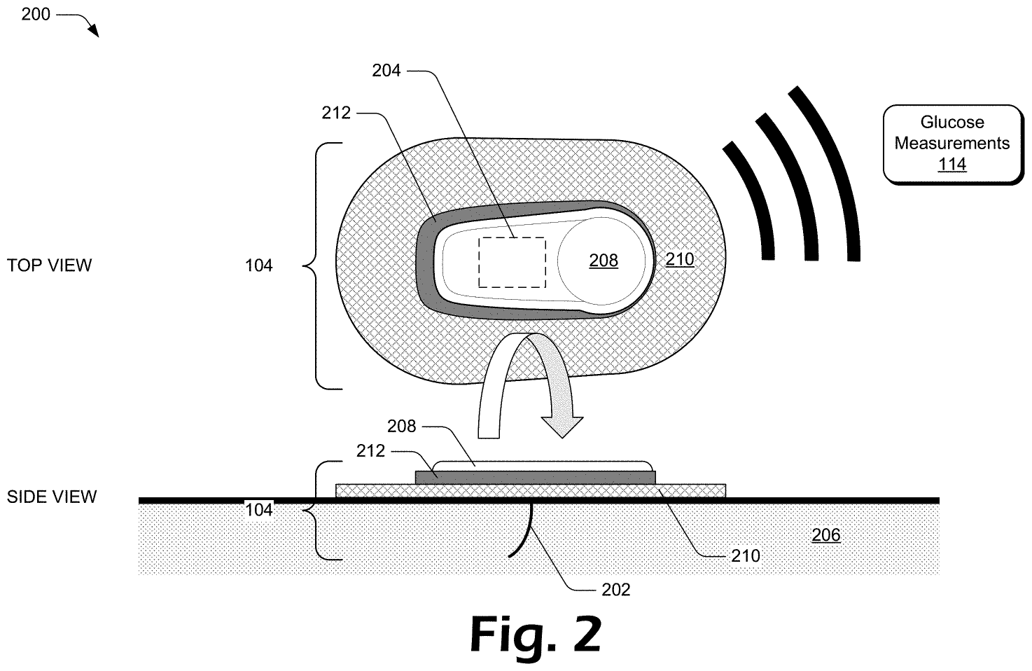

In one or more implementations, the wearable glucose monitoring device 104 is a continuous glucose monitoring (“CGM”) system. As used herein, the term “continuous” used in connection with glucose monitoring may refer to an ability of a device to produce measurements substantially continuously, such that the device may be configured to produce the glucose measurements 114 at intervals of time (e.g., every hour, every 30 minutes, every 5 minutes, and so forth), responsive to establishing a communicative coupling with a different device (e.g., when a computing device establishes a wireless connection with the wearable glucose monitoring device 104 to retrieve one or more of the measurements), and so forth This functionality along with further aspects of the wearable glucose monitoring device 104's configuration are discussed in more detail in relation to FIG. 2.

Additionally, the wearable glucose monitoring device 104 transmits the glucose measurements 114 to the computing device 106, such as via a wireless connection. The wearable glucose monitoring device 104 may communicate these measurements in real-time, e.g., as they are produced using a glucose sensor. Alternately or in addition, the wearable glucose monitoring device 104 may communicate the glucose measurements 114 to the computing device 106 at set time intervals. For example, the wearable glucose monitoring device 104 may be configured to communicate the glucose measurements 114 to the computing device 106 every five minutes (as they are being produced).

Certainly, an interval at which the glucose measurements 114 are communicated may be different from the examples above without departing from the spirit or scope of the described techniques. The measurements may be communicated by the wearable glucose monitoring device 104 to the computing device 106 according to other bases in accordance with the described techniques, such as based on a request from the computing device 106. Regardless, the computing device 106 may maintain the glucose measurements 114 of the person 102 at least temporarily, e.g., in computer-readable storage media of the computing device 106.

Although illustrated as a mobile device (e.g., a mobile phone), the computing device 106 may be configured in a variety of ways without departing from the spirit or scope of the described techniques. By way of example and not limitation, the computing device 106 may be configured as a different type of mobile device (e.g., a wearable device or tablet device). In one or more implementations, the computing device 106 may be configured as a dedicated device associated with the glucose monitoring platform 110, e.g., with functionality to obtain the glucose measurements 114 from the wearable glucose monitoring device 104, perform various computations in relation to the glucose measurements 114, display information related to the glucose measurements 114 and the glucose monitoring platform 110, communicate the glucose measurements 114 to the glucose monitoring platform 110, and so forth.

Additionally, the computing device 106 may be representative of more than one device in accordance with the described techniques. In one or more scenarios, for instance, the computing device 106 may correspond to both a wearable device (e.g., a smart watch) and a mobile phone. In such scenarios, both of these devices may be capable of performing at least some of the same operations, such as to receive the glucose measurements 114 from the wearable glucose monitoring device 104, communicate them via the network 112 to the glucose monitoring platform 110, display information related to the glucose measurements 114, and so forth. Alternately or in addition, different devices may have different capabilities that other devices do not have or that are limited through computing instructions to specified devices.

In the scenario where the computing device 106 corresponds to a separate smart watch and a mobile phone, for instance, the smart watch may be configured with various sensors and functionality to measure a variety of physiological markers (e.g., heartrate, heartrate variability, breathing, rate of blood flow, and so on) and activities (e.g., steps or other exercise) of the person 102. In this scenario, the mobile phone may not be configured with these sensors and functionality, or it may include a limited amount of that functionality—although in other scenarios a mobile phone may be able to provide the same functionality. Continuing with this particular scenario, the mobile phone may have capabilities that the smart watch does not have, such as a camera to capture images of meals for meal logging and an amount of computing resources (e.g., battery and processing speed) that enables the mobile phone to more efficiently carry out computations in relation to the glucose measurements 114. Even in scenarios where a smart watch is capable of carrying out such computations, computing instructions may limit performance of those computations to the mobile phone so as not to burden both devices and to utilize available resources efficiently. To this extent, the computing device 106 may be configured in different ways and represent different numbers of devices than discussed herein without departing from the spirit and scope of the described techniques.

In accordance with the discussed techniques, the computing device 106 is configured to implement meal and activity (i.e., event) logging with a glucose monitoring user interface. In the environment 100, the computing device 106 includes glucose monitoring application 116 and storage device 118. Here, the glucose monitoring application 116 includes the event logging module 120. In the illustrated environment 100, the glucose measurements 114 and one or more event logs 122 (e.g., meal and/or activity logs) are shown stored in storage device 118 of the computing device 106. The storage device 118 may represent one or more databases and also other types of storage capable of storing the glucose measurements 114 and the event logs 122. In one or more implementations, the glucose measurements 114 and/or the event logs 122 may be stored at least partially remote from the computing device 106, e.g., in storage of the glucose monitoring platform 110, and retrieved or otherwise accessed in connection with meal and activity logging with glucose monitoring user interface. For instance, the glucose measurements 114 and/or the event logs 122 may be generally stored in storage of the glucose monitoring platform 110 along with the glucose measurements and/or the event logs 122 of the user population 108, and some of that data may be retrieved or otherwise accessed on an as-needed basis to display a glucose monitoring user interface with meal and activity logging information.

The storage device 118 may also store a variety of other data. In accordance with the described techniques, for instance, the person 102 corresponds to a user of at least the glucose monitoring platform 110 and may also be a user of one or more other, third party service providers. To this end, the person 102 may be associated with a username and be required, at some time, to provide authentication information (e.g., password or biometric data) to access the glucose monitoring platform 110 using the username. This information, along with other information about the user, may be maintained in the storage device 118, including, for example, application settings (e.g., of the glucose monitoring application 116), device usage settings (e.g., information sharing settings), demographic information describing the person 102, information about a health care provider, payment information, prescription information, determined health indicators, user preferences, account information for other service provider systems (e.g., a service provider associated with a wearable, social networking systems, telemedicine services, and so on), and so forth.

Broadly speaking, the glucose monitoring application 116 is configured to support interactions with a user that enable glucose of the user (or a different user) to be monitored. In one or more implementations, the glucose monitoring platform 110 is also leveraged to monitor the glucose of the user and support interactions via the glucose monitoring application 116. As noted above, for instance, the glucose monitoring platform 110 may be configured to store data, such as the glucose measurements 114 and the event logs 122 associated with a user (e.g., the person 102) and/or users of the user population 108. The glucose monitoring platform 110 may also provide updates and/or additions to the glucose monitoring application 116. Further still, the glucose monitoring platform 110 may train, maintain, and/or deploy algorithms (e.g., machine learning algorithms) to generate predictions in connection with monitoring glucose, such as by using the wealth of data collected from the person 102 and the users of the user population 108. One or more such algorithms may require an amount of computing resources that exceeds the resources of typical, personal computing devices, e.g., mobile phones, laptops, tablet devices, and wearables, to name just a few. Nonetheless, the glucose monitoring platform 110 may include or otherwise have access to the amount of resources needed to operate such algorithms, e.g., cloud storage, server devices, virtualized resources, and so forth. The glucose monitoring platform 110 may provide a variety of resources that the glucose monitoring application 116 leverages in connection with enabling glucose of users to be monitored.

In accordance with the described techniques, the glucose monitoring application 116 is configured to generate and cause display of one or more user interfaces that present information related to glucose monitoring. The glucose monitoring application 116 may generate user interface 124, for instance, and cause its display via a display device of the computing device 106. By way of example, the glucose monitoring application 116 may generate the user interface 124 to include one or more of the glucose measurements 114, such as a “trace” of the glucose measurements 114 over one or more intervals of time. These intervals of time may precede a current time, such as a last 24 hours, a last 12 hours, a last 6 hours, and a last 3 hours, to name just a few. Indeed, the glucose monitoring application 116 may cause display of the glucose measurements 114 over a variety of intervals without departing from the spirit or scope of the described techniques, including one or more predicted glucose measurements subsequent to a current time.

As discussed above and below, the glucose monitoring application 116 is further configured to cause display of meal and/or activity logging information (i.e., event information) along with the glucose measurements 114 via the user interface 124. For example, the glucose monitoring application 116 is configured to cause display of one or more event representations 126 on the user interface 124 with a glucose graph 128, e.g., that plots one or more of the glucose measurements 114 over time. In order to display the event representations 126, the glucose monitoring application 116 may use the event logging module 120.

The event logging module 120 is configured to enable events (e.g., meals or activities) to be added to the event logs 122, including by receiving data describing or otherwise associated with events. Based on the data received, for instance, the event logging module 120 may configure the individual event representations 126 differently for display, e.g., to include an image captured in association with the respective event, to include a visual indication that is modifiable based on a characteristic of the event, to associate a display position in the glucose graph 128 with the event (that corresponds to a time of the event), and so forth.

The event logging module 120 may also provide supplemental information or generate supplemental information portions for display as part of the user interface 124, such as information about a meal to supplement a visual representation of the meal displayed on the glucose graph 128. In one or more implementations, the event logging module 120 may also be configured to import event data from other applications to generate and display the event representations 126 via the user interface 124 with the glucose graph 128, such as to import event data from a meal logging application or a health tracking application (or device) that is separate from the glucose monitoring application 116. The event logging module 120 may perform a variety of actions in connection with logging events and causing display of event information (e.g., event representations) via a glucose monitoring interface without departing from the spirit or scope of the techniques described herein. In the context of measuring glucose, e.g., continuously, and obtaining data describing such measurements, consider the following discussion of FIG. 2.

FIG. 2 depicts an example of an implementation 200 of the wearable glucose monitoring device 104 of FIG. 1 in greater detail. In particular, the illustrated example 200 includes a top view and a corresponding side view of the wearable glucose monitoring device 104. It is to be appreciated that the wearable glucose monitoring device 104 may vary in implementation from the following discussion in various ways without departing from the spirit or scope of the described techniques. As noted above, for instance, meal and activity (i.e., event) logging with a glucose monitoring user interface may be performed in connection with other types of devices for glucose monitoring, such as non-wearable devices (e.g., blood glucose meters requiring finger sticks), patches, and so forth.

In this example 200, the wearable glucose monitoring device 104 is illustrated to include a sensor 202 and a sensor module 204. Here, the sensor 202 is depicted in the side view having been inserted subcutaneously into skin 206, e.g., of the person 102. The sensor module 204 is depicted in the top view as a dashed rectangle. The wearable glucose monitoring device 104 also includes a transmitter 208 in the illustrated example 200. Use of the dashed rectangle for the sensor module 204 indicates that it may be housed or otherwise implemented within a housing of the transmitter 208. In this example 200, the wearable glucose monitoring device 104 further includes adhesive pad 210 and attachment mechanism 212.

In operation, the sensor 202, the adhesive pad 210, and the attachment mechanism 212 may be assembled to form an application assembly, where the application assembly is configured to be applied to the skin 206 so that the sensor 202 is subcutaneously inserted as depicted. In such scenarios, the transmitter 208 may be attached to the assembly after application to the skin 206 via the attachment mechanism 212. Additionally or alternately, the transmitter 208 may be incorporated as part of the application assembly, such that the sensor 202, the adhesive pad 210, the attachment mechanism 212, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, the user initiated application of the wearable glucose monitoring device 104 is nearly painless and does not require the withdrawal of blood. Moreover, the automatic sensor applicator generally enables the person 102 to embed the sensor 202 subcutaneously into the skin 206 without the assistance of a clinician or healthcare provider.

The application assembly may also be removed by peeling the adhesive pad 210 from the skin 206. It is to be appreciated that the wearable glucose monitoring device 104 and its various components as illustrated are simply one example form factor, and the wearable glucose monitoring device 104 and its components may have different form factors without departing from the spirit or scope of the described techniques.

In operation, the sensor 202 is communicatively coupled to the sensor module 204 via at least one communication channel which can be a wireless connection or a wired connection. Communications from the sensor 202 to the sensor module 204 or from the sensor module 204 to the sensor 202 can be implemented actively or passively and these communications can be continuous (e.g., analog) or discrete (e.g., digital).

The sensor 202 may be a device, a molecule, and/or a chemical which changes or causes a change in response to an event which is at least partially independent of the sensor 202. The sensor module 204 is implemented to receive indications of changes to the sensor 202 or caused by the sensor 202. For example, the sensor 202 can include glucose oxidase which reacts with glucose and oxygen to form hydrogen peroxide that is electrochemically detectable by the sensor module 204 which may include an electrode. In this example, the sensor 202 may be configured as or include a glucose sensor configured to detect analytes in blood or interstitial fluid that are indicative of glucose level using one or more measurement techniques. In one or more implementations, the sensor 202 may also be configured to detect analytes in the blood or the interstitial fluid that are indicative of other markers, such as lactate levels, which may improve accuracy in generating various predictions in connection with glucose monitoring. Additionally or alternately, the wearable glucose monitoring device 104 may include additional sensors to the sensor 202 to detect those analytes indicative of the other markers.

In another example, the sensor 202 (or an additional sensor of the wearable glucose monitoring device 104—not shown) can include a first and second electrical conductor and the sensor module 204 can electrically detect changes in electric potential across the first and second electrical conductor of the sensor 202. In this example, the sensor module 204 and the sensor 202 are configured as a thermocouple such that the changes in electric potential correspond to temperature changes. In some examples, the sensor module 204 and the sensor 202 are configured to detect a single analyte, e.g., glucose. In other examples, the sensor module 204 and the sensor 202 are configured to detect multiple analytes, e.g., sodium, potassium, carbon dioxide, and glucose. Alternately or additionally, the wearable glucose monitoring device 104 includes multiple sensors to detect not only one or more analytes (e.g., sodium, potassium, carbon dioxide, glucose, and insulin) but also one or more environmental conditions (e.g., temperature). Thus, the sensor module 204 and the sensor 202 (as well as any additional sensors) may detect the presence of one or more analytes, the absence of one or more analytes, and/or changes in one or more environmental conditions.

In one or more implementations, the sensor module 204 may include a processor and memory (not shown). The sensor module 204, by leveraging the processor, may generate the glucose measurements 114 based on the communications with the sensor 202 that are indicative of the above-discussed changes. Based on these communications from the sensor 202, the sensor module 204 is further configured to generate communicable packages of data that include at least one glucose measurement 114. In one or more implementations, the sensor module 204 may configure those packages to include additional data, including, by way of example and not limitation, a sensor identifier, a sensor status, temperatures that correspond to the glucose measurements 114, measurements of other analytes that correspond to the glucose measurements 114, and so forth. It is to be appreciated that such packets may include a variety of data in addition to at least one glucose measurement 114 without departing from the spirit or scope of the described techniques.

In implementations where the wearable glucose monitoring device 104 is configured for wireless transmission, the transmitter 208 may transmit the glucose measurements 114 wirelessly as a stream of data to a computing device. Alternately or additionally, the sensor module 204 may buffer the glucose measurements 114 (e.g., in memory of the sensor module 204 and/or other physical computer-readable storage media of the wearable glucose monitoring device 104) and cause the transmitter 208 to transmit the buffered glucose measurements 114 later at various intervals, e.g., time intervals (every second, every thirty seconds, every minute, every five minutes, every hour, and so on), storage intervals (when the buffered glucose measurements 114 reach a threshold amount of data or a number of measurements), and so forth.

Having considered an example of an environment and an example of a wearable glucose monitoring device, consider now a discussion of some examples of details of the techniques for meal and activity logging with a glucose monitoring user interface in a digital medium environment in accordance with one or more implementations.

Meal and Activity Logging with a Glucose Monitoring UI

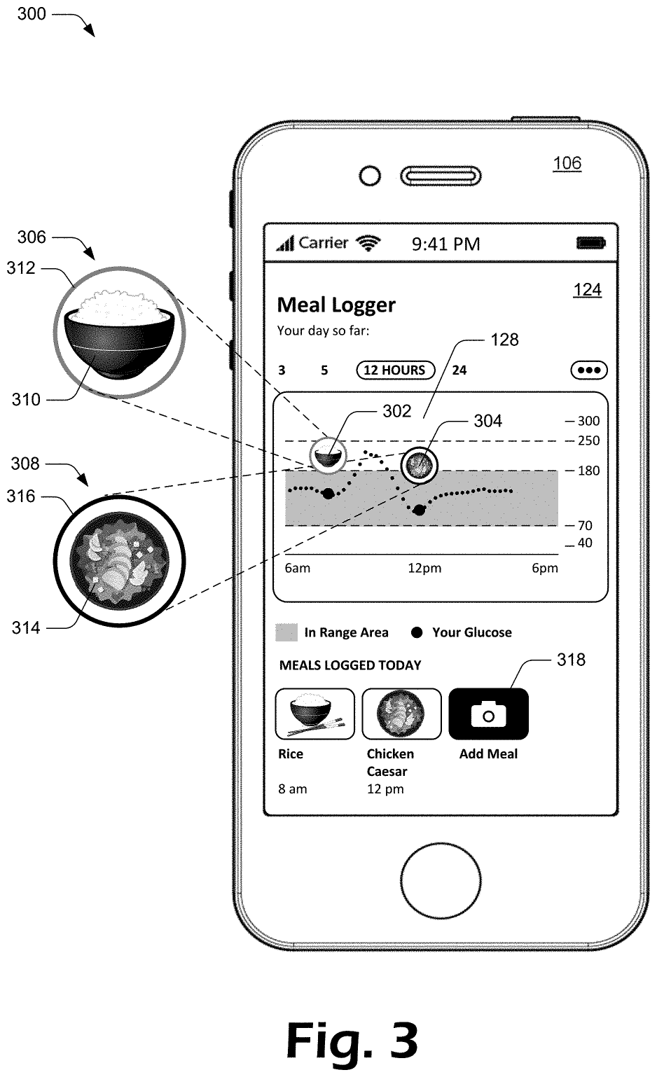

FIG. 3 depicts an example 300 of a user interface displaying meal logging visual representations with glucose measurements. The illustrated example 300 includes from FIG. 1 an example of the computing device 106 displaying an example of the user interface 124 via a display device, e.g., a touchscreen.

Here, the user interface 124 includes the glucose graph 128, which plots one or more of the glucose measurements 114 (e.g., of the person 102) over time. In the illustrated example 300, the time period over which the plotted glucose measurements 114 are displayed in the glucose graph 128 is a 12-hour period. The user interface 124 also includes selectable elements that are selectable to display the glucose measurements 114 plotted over different time periods, including a 3-hour time period, a 5-hour time period, and a 24-hour time period. In one or more implementations, these time periods correspond to time periods that precede a current time, e.g., to enable a user to review historical events logged previously in connection with their glucose. In this way, the user interface 124 may visually convey the historical effects of different meals eaten and/or activities engaged in on a user's glucose. In any case, the glucose monitoring application 116 may plot the glucose measurements 114 on the glucose graph 128 over different periods of time without departing from the spirit or scope of the techniques described herein.

In addition to the plotted glucose measurements 114, the user interface 124 includes first and second meal representations 302, 304, which are presented on the glucose graph 128 in the illustrated example 300. In this example 300, the meal representations 302 and 304 can be seen to overlay the glucose graph 128 which enables the user to visually identify the effect that the respective meals had on the user's glucose. In accordance with the described techniques, the event logging module 120 may cause the first and second meal representations 302, 304 to be presented at positions on the glucose graph 128 that correspond to times associated with the respective meals logged. The times associated with meal representations may be included in the meal data received by the event logging module 120 to log meals and cause display of the meal representations along with glucose measurements.

Notably, the presentation of the meal representations 302, 304 in conjunction with the glucose graph 128 provides a visual correlation between the respective meals consumed by the user and the impact of the meals on the glucose levels of the user over the course of the subsequent minutes or hours. This visual association between meals and glucose measurements can educate a user about which meals help the user meet his or her health goals (e.g., help keep the user's glucose measurements within a target range) and which events may be preventing the user from meeting his or her health goals (e.g., cause the user's glucose measurements to be outside the target range). Doing so supports self-learning regarding the impact of various meals, and the timing of meals, on glucose which enables users to connect the dots between meals and their glucose levels, and thus leads the user to make meaningful every day changes for their long-term health, such as by eating different meals.

In FIG. 3, for example, the glucose measurements on glucose graph 128 can be seen to rise sharply after the consumption of the bowl of white rice as compared to small rise in glucose measurements that occurred after consumption of the salad. Based on this visual correlation between glucose measurements and meals, therefore, the user can learn that white rice causes a sharp rise in glucose measurements which takes the user out of range. The user may thus begin to choose salads over white rice in order to control their glucose values.

The event logging module 120 may associate a time with a meal by identifying a time (e.g., via a timestamp) when a photo of the meal is captured, e.g., using a camera of the computing device 106. In this case, the meal data received by the event logging module 120 would include both the photo of the meal as well as the timestamp without the user having to provide any additional manual input other than initiating the capture of the photo. In one or more implementations, the event logging module 120 may also automatically determine and associate a location at which the meal was consumed with the meal. Doing so may enable tracking of different types of meals based on location, e.g., meals consumed at home versus meals consumed at restaurants. In some cases the location information may also enable accurate nutrition information tracking, e.g., a cheeseburger consumed at a location associated with a particular restaurant may cause nutrition information to be obtained from online nutrition information associated with the particular restaurant. Alternatively or additionally, the event logging module 120 may receive a user entry of a time for a meal via one or more instrumentalities, e.g., text entry, dropdown box, one or more time scroll wheels, or tapping on the glucose graph to log a meal, to name just a few. In this case, for instance, the user could snap a photo of the meal and then provide additional user input to associate a time with the meal. Further still, the event logging module 120 may automatically select a time that corresponds to a user input to log a meal as a default time for the meal representation. The event logging module 120 may enable this default time to be modified by a user via one or more instrumentalities, such as those mentioned just above. Indeed, the event logging module 120 may associate times with meals in a variety of ways without departing from the spirit or scope of the techniques described herein.

The event logging module 120 may also enable users to “retroactively” log meals or activities. For example, in the course of a day, users may forget to log certain meals or activities in real-time. In this scenario, the event logging module 120 may provide functionality which enables the user to log meals and activities retroactively, e.g., at the end of the day or in the morning the following day. In one or more implementations, the glucose graph is selectable by the user in order to associate events logged retroactively with a specific time on the glucose graph. For example, if the user recalls eating a chicken salad at 1 pm, then the user can retroactively tap the glucose graph at a position on the glucose graph which corresponds to a time of 1 pm, and then provide input to log the chicken salad, e.g., by inputting a photo of the chicken salad taken earlier that day, selecting the chicken salad from a list, providing text input to log the chicken salad, and so forth. In these cases, the input selecting a particular time from the glucose graph (e.g., via a tap input) can be used by the event logging module 120 to automatically determine and associate a time with the retroactively logged meal without requiring any further input by the user. In other words, by tapping on the glucose graph to select a position corresponding to 1 pm, the event logging module automatically logs the chicken salad with the time of 1 pm. Notably, the glucose graph may also provide a visual clue to the user regarding the time of a meal when the user is logging events retroactively. For example, if the user remembers eating a bowl of white rice in the afternoon but cannot recall the exact time at which this meal was consumed, the user can open the glucose monitoring application to view the glucose graph, and notice a spike in glucose level around 2 pm. In this case, the spike of glucose around 2 pm provides a visual clue which enables the user to determine that the meal was likely consumed at approximately 2 pm. Thus, the user can log the meal at 2 pm, e.g., by tapping the glucose graph at a position corresponding to 2 pm and providing input to log the bowl of white rice.

In addition to presenting the first and second meal representations 302, 304 at positions corresponding to times associated with the logged meals, the event logging module 120 may cause the user interface 124 to include additional visual elements to visually emphasize association of the first and second meal representations 302, 304 with the plotted glucose measurements on the glucose graph 128. For example, the event logging module 120 may cause one or more of the glucose measurements that correspond to a time associated with a meal representation to be visually emphasized, e.g., the plotted glucose measurement(s) may be enlarged relative to measurements not associated with a meal, have a different color or different colors than measurements not associated with a meal, have a different shape than measurements not associated with a meal, and so forth. Alternatively or additionally, the event logging module 120 may add a visual marker in line with the plotted glucose measurements at the time associated with the respective meal representation. By “in line” it is meant that the visual marker may be positioned at the time associated with the meal (x-value) and/or approximately at a glucose value (y-value) interpolated from an immediately preceding and an immediately subsequent glucose measurement, e.g., when the time associated with the meal is between times of two glucose measurements. These visual markers may be emphasized in manners similar to those discussed just above for emphasizing glucose measurements.

The illustrated example 300 also includes an expanded view 306 of the first meal representation 302 and an expanded view 308 of the second meal representation 304. The expanded views 306, 308 are included to discuss example details of the first and second meal representations 302, 304 as they may be displayed on the glucose graph 128. As depicted in the expanded view 306, the first meal representation 302 includes an image 310 of the logged meal. The image 310 may correspond to a photograph captured of the logged meal, e.g., by a camera of the computing device 106, or may correspond to an icon representative of the logged meal, e.g., selected by the user as discussed below or selected automatically by the event logging module 120. The image 310 may be selected automatically by the event logging module 120, for instance, when meal data is received at least in part via voice commands. It is to be appreciated that an image used with a meal representation may be selected in a variety of ways without departing from the spirit or scope of the described techniques—in some implementations meal representations may be text based and not include an image corresponding to the meal as discussed below.

In the expanded view 306, the first meal representation 302 also includes visual indication 312. In general, the visual indication 312 indicates a characteristic of the meal. By way of example and not limitation, the visual indication 312 may indicate a classification of the corresponding meal as having generally ‘good’, ‘bad’, or ‘neutral’ healthfulness. Alternatively or additionally, the visual indication 312 may indicate an amount or estimated amount of carbohydrates of the meal, such as low, medium, or high. Certainly, the visual indication 312 may convey information about various characteristics of a meal in the spirit or scope of the described techniques. Alternately or additionally, the visual indication 312 may be based on the historical impact of a particular food on glucose for the particular user. For example, if a particular type of cereal historically has a negative effect on the user's glucose, then the visual indication 312 may indicate “bad” healthfulness. In contrast, if the same type of cereal historically has a neutral effect on the glucose of a different user, then the visual indication 312 may indicate “neutral” healthfulness for the different user. Additionally, a meal representation may include multiple visual indications (e.g., multiple rings) to visually indicate multiple characteristics about a meal.

To visually indicate a characteristic, the visual indication 312 may have different visual properties depending on different characteristics. For instance, different colors of the visual indication 312 may be indicative of different characteristics. Given the above example in which a meal may be associated with a ‘good’, ‘bad’, or ‘neutral’ healthfulness, the visual indication 312 may have a first color if it is associated with ‘good’ healthfulness (e.g., green), a second color if it is associated with ‘bad’ healthfulness (e.g., red), or a third color if it is associated with ‘neutral’ healthfulness (e.g., yellow). In this example 300, the visual indication 312 is illustrated as a ring (e.g., a colored ring) surrounding the first meal representation 302. A visual indication of a meal representation may be configured in a variety of ways to convey information (e.g., a characteristic) about a respective meal.

As depicted in the expanded view 308, the second meal representation 304 includes an image 314 of the logged meal and a visual indication 316 surrounding the second meal representation 304. The image 314 and the visual indication 316 may be configured in a similar manner to the image 310 and the visual indication 312 as discussed in relation to the expanded view 306 of the first meal representation 302.

In this example 300, the user interface 124 also includes meal logging control 318, which is selectable to log a meal and display a meal representation on the glucose graph 128 at a position corresponding to a time of the logged meal. In this example, the meal logging control is depicted with an icon of a camera to represent that selection of the meal logging control 318 initiates an interface for capturing a photo of a meal. Alternately, however, the meal logging control 318 could be selected in order to initiate the display of an interface for receiving user input, such as to receive text entry describing a meal or to receive user input to select a meal from a displayed list of common meals.

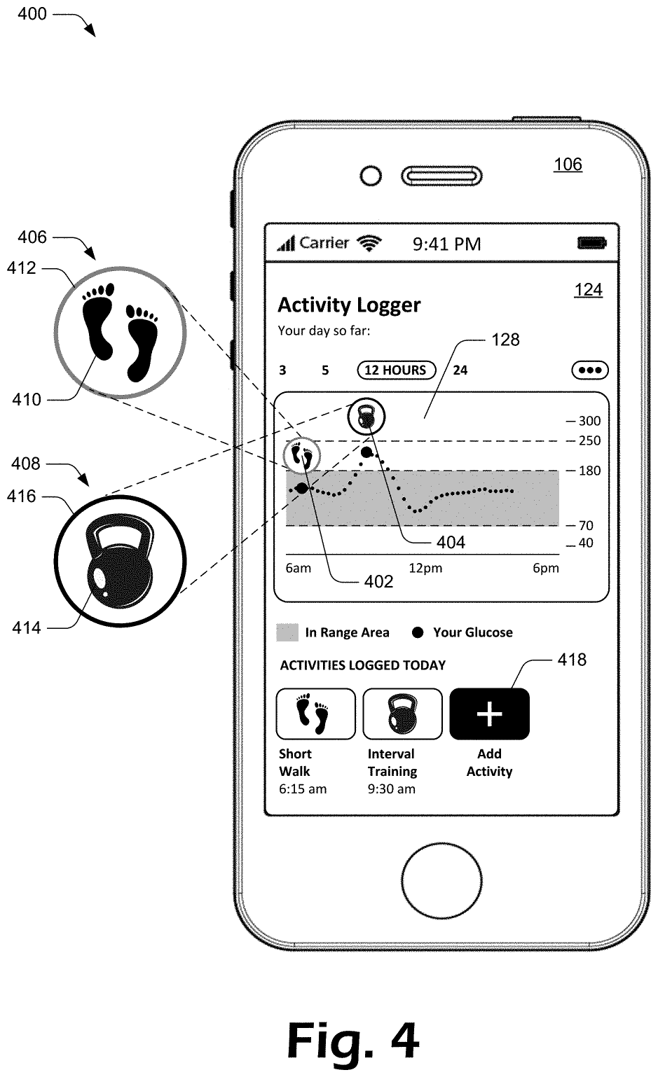

Although illustrated as a selectable user interface element that is displayed, the meal logging control 318 may be configured in different ways to enable a meal to be logged and a meal representation to be displayed on the glucose graph 128 at a position corresponding to the time of the logged meal. For instance, the meal logging control 318 may be activated by voice command, such as by the computing device 106 or another computing device receiving a spoken command to log a meal. In this case, for example, the user could simply speak a voice command such as “log white rice” in order to have the white rice logged automatically without any additional user input. In one or more implementations, the event logging module 120 may be configured to log meals both responsive to selection of a displayed user interface element and responsive to voice commands. The event logging module 120 may be configured to log meals in additional or different ways in accordance with the described techniques. Consider now the following discussion of FIG. 4 in connection with logging activities.

FIG. 4 depicts an example 400 of a user interface displaying activity logging visual representations with glucose measurements. The illustrated example 400 includes from FIG. 1 an example of the computing device 106 displaying an example of the user interface 124 via a display device, e.g., a touchscreen.

Like in the illustrated example 300, in this example 400 the user interface 124 includes the glucose graph 128, which plots one or more of the glucose measurements 114 (e.g., of the person 102) over time. Again, the time period over which the plotted glucose measurements 114 are displayed in the glucose graph 128 is a 12-hour period. Nevertheless, the glucose monitoring application 116 may plot the glucose measurements 114 on the glucose graph 128 over different periods of time without departing from the spirit or scope of the techniques described herein.

In contrast to the example 300, the user interface 124 in this example 400 includes first and second activity representations 402, 404 presented on the glucose graph 128. In accordance with the described techniques, the event logging module 120 may cause the first and second activity representations 402, 404 to be presented at positions on the glucose graph 128 that correspond to times associated with the respective activities logged. The times associated with activity representations may comprise at least a portion of activity data received by the event logging module 120 to log an activity and cause display of the activity representations along with glucose measurements.

By way of example, the event logging module 120 may associate a time with an activity representation by identifying a time (e.g., via a timestamp) of an activity from an activity tracking device (e.g., a smart watch, chest strap, or the computing device 106 when used as one) or an activity tracking application. Alternatively or additionally, the event logging module 120 may receive a user entry of a time for an activity via one or more instrumentalities, e.g., text entry, dropdown box, or one or more time scroll wheels, to name just a few. Further still, the event logging module 120 may automatically select a time that corresponds to a user input to log an activity as a default time for the activity representation. The event logging module 120 may enable this default time to be modified by a user via one or more instrumentalities, such as those mentioned just above. Indeed, the event logging module 120 may associate times with activities in a variety of ways without departing from the spirit or scope of the techniques described herein.

In addition to presenting the first and second activity representations 402, 404 at positions corresponding to times associated with the logged activities, the event logging module 120 may cause the user interface 124 to include additional visual elements to visually emphasize association of the first and second activity representations 402, 404 with the plotted glucose measurements on the glucose graph 128. For example, the event logging module 120 may cause one or more of the glucose measurements that correspond to a time associated with an activity representation to be visually emphasized, e.g., the plotted glucose measurement(s) may be enlarged relative to measurements not associated with an activity, have a different color or different colors than measurements not associated with an activity, have a different shape than measurements not associated with an activity, and so forth. Alternatively or additionally, the event logging module 120 may add a visual marker in line with the plotted glucose measurements at the time associated with the respective activity representation. By “in line” it is meant that the visual marker may be positioned at the time associated with the activity (x-value) and/or approximately at a glucose value (y-value) interpolated from an immediately preceding and an immediately subsequent glucose measurement, e.g., when the time associated with the activity is between times of two glucose measurements. These visual markers may be emphasized in manners similar to those discussed just above for emphasizing glucose measurements.

The illustrated example 400 also includes an expanded view 406 of the first activity representation 402 and an expanded view 408 of the second activity representation 404. The expanded views 406, 408 are included to discuss example details of the first and second activity representations 402, 404 as they may be displayed on the glucose graph 128. As depicted in the expanded view 406, the first activity representation 402 includes an image 410 of the logged activity. The image 410 may correspond to a photograph captured in connection with the logged activity, e.g., by a camera of the computing device 106, or may correspond to an icon representative of the logged activity, e.g., selected by the user as discussed below or selected automatically by the event logging module 120. The image 410 may be selected automatically by the event logging module 120, for instance, when activity data is received at least in part via voice commands or from an activity tracking device or an activity tracking application. It is to be appreciated that an image used with an activity representation may be selected in a variety of ways without departing from the spirit or scope of the described techniques—in some implementations activity representations may be text based and not include an image corresponding to the activity.

In the expanded view 406, the first activity representation 402 also includes visual indication 412. In general, the visual indication 412 indicates a characteristic of the activity. By way of example and not limitation, the visual indication 412 may indicate a classification of how the user feels during the activity, such as whether the user feels generally ‘good’, ‘bad’, or ‘neutral’ during the activity. Alternatively or additionally, the visual indication 412 may indicate an amount or estimated amount of calories burned during the activity, such as low, medium, or high. Alternatively or additionally, the visual indication 412 may indicate a VO₂ max (e.g., maximal oxygen consumption) of the user during the activity, such as low, medium, or high consumption. Alternatively or additionally, the visual indication 412 may indicate an intensity or exertion rating of the user during the activity, such as low, medium, or high intensity. Such an intensity or exertion rating could be determined based on data obtained from an activity tracker (e.g., intensity determined based on the user's heart rate during the activity) or self-perceived exertion by the user, e.g., the user could rate the level of exertion after the activity on a scale of 1-10. Associating the intensity or exertion of an activity with the user's glucose, whether self-perceived or objectively measured by an activity tracker, enables the user to view the effects of different levels of intensity or exertion on glucose. Examples of additional characteristics may include, by way of example and not limitation, an average and/or maximum heart rate of the user during the activity, an amount of work performed by the user during the activity, power output during the activity, a score of the user in connection with the activity (e.g., a golf score), one or more distances associated with the activity (e.g., a distance traversed or amount of vertical gained), and so forth. Certainly, the visual indication 412 may convey information about various characteristics of a meal in the spirit or scope of the described techniques. Additionally, an activity representation may include multiple visual indications (e.g., multiple rings) to visually indicate multiple characteristics about an activity.

To visually indicate a characteristic, the visual indication 412 may have different visual properties depending on different characteristics. For instance, different colors of the visual indication 412 may be indicative of different characteristics. Given the above example in which an activity may be associated with a generally ‘good’, ‘bad’, or ‘neutral’ feeling of the user during an activity, the visual indication 412 may have a first color if it is associated with ‘good’ feeling (e.g., green), a second color if it is associated with ‘bad’ feeling (e.g., red), or a third color if it is associated with ‘neutral’ feeling (e.g., yellow). In this example 400, the visual indication 412 is illustrated as a ring (e.g., a colored ring) surrounding the first activity representation 402. A visual indication of an activity representation may be configured in a variety of ways to convey information (e.g., a characteristic) about a respective activity. As mentioned above, an activity representation may also include multiple visual indications.

As depicted in the expanded view 408, the second activity representation 404 includes an image 414 of the logged activity and a visual indication 416 surrounding the second activity representation 404. The image 414 and the visual indication 416 may be configured in a similar manner to the image 410 and the visual indication 412 as discussed in relation to the expanded view 406 of the first activity representation 402.

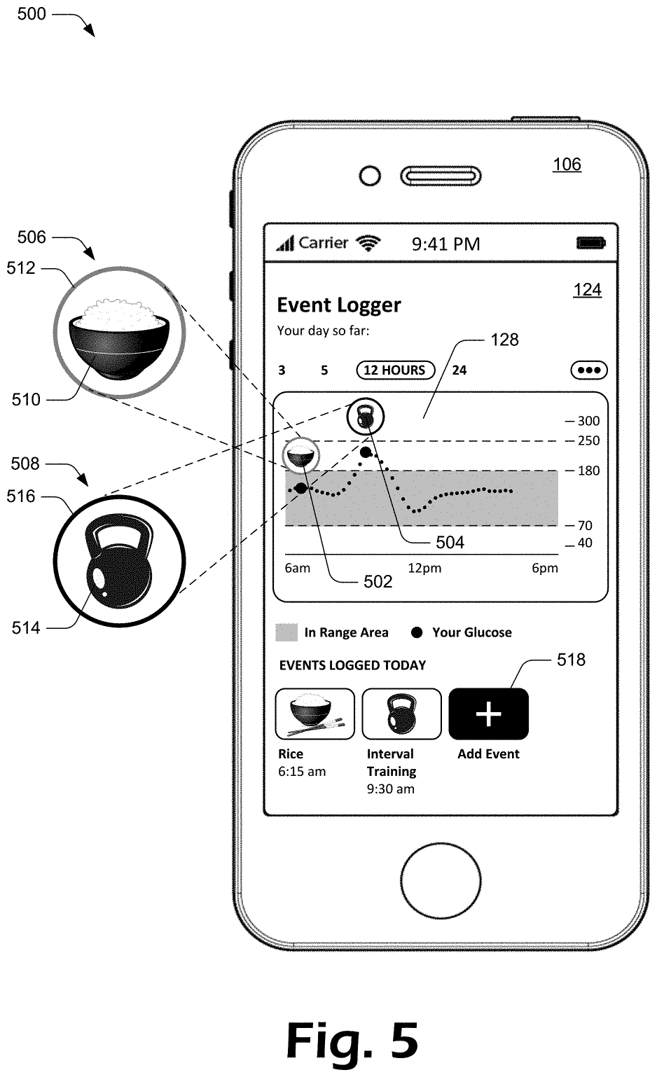

In this example 400, the user interface 124 also includes activity logging control 418, which is selectable to log an activity and display an activity representation on the glucose graph 128 at a position corresponding to a time of the logged activity. Although illustrated as a selectable user interface element that is displayed, the activity logging control 418 may be configured in different ways to enable an activity to be logged and an activity representation to be displayed on the glucose graph 128 at a position corresponding to the time of the logged activity. For instance, the activity logging control 418 may be activated by voice command, such as by the computing device 106 or another computing device (e.g., a smart watch or voice assistant) receiving a spoken command to log an activity. In one or more implementations, the event logging module 120 may be configured to log activities both responsive to selection of a displayed user interface element and responsive to voice commands. The event logging module 120 may be configured to log activities in additional or different ways in accordance with the described techniques. Consider now the following discussion of FIG. 5 in connection with logging events (e.g., meals and/or activities).

FIG. 5 depicts an example 500 of a user interface displaying event logging visual representations with glucose measurements. The illustrated example 500 includes from FIG. 1 an example of the computing device 106 displaying an example of the user interface 124 via a display device, e.g., a touchscreen.

Like in the illustrated examples 300, 400, in this example 500 the user interface 124 includes the glucose graph 128, which plots one or more of the glucose measurements 114 (e.g., of the person 102) over time. Again, the time period over which the plotted glucose measurements 114 are displayed in the glucose graph 128 is a 12-hour period. Nevertheless, the glucose monitoring application 116 may plot the glucose measurements 114 on the glucose graph 128 over different periods of time without departing from the spirit or scope of the techniques described herein.

Here, the user interface 124 in this example 500 includes first and second event representations 502, 504 presented on the glucose graph 128. In contrast to the above discussed examples 300, 400, this example 500 depicts both a meal representation (e.g., the first event representation 502) and an activity representation (e.g., the second event representation 504). Thus, in one or more implementations, a combination of meals and activities may be represented on the glucose graph 128. It is to be appreciated that other events may also be represented on the glucose graph 128 by event representations, such as sleep, health events, medicine administration, stressful events (e.g., work projects and/or tasks), and vacation, to name just a few.

Regardless of the particular type of event, the event logging module 120 is configured to cause the first and second event representations 502, 504 to be presented at positions on the glucose graph 128 that correspond to times associated with the respective events logged. The times associated with event representations may comprise at least a portion of event data received by the event logging module 120 to log an event and cause display of the event representations along with glucose measurements. The event logging module 120 may associate a time with an event, for example, by identifying a time of the event (e.g., a meal or an activity) as discussed in detail above. It is to be appreciated that the event logging module 120 may associate times with events in a variety of ways without departing from the spirit or scope of the techniques described herein.

In addition to presenting the first and second event representations 502, 504 at positions corresponding to times associated with the logged events, the event logging module 120 may cause the user interface 124 to include additional visual elements to visually emphasize association of the first and second event representations 502, 504 with the plotted glucose measurements on the glucose graph 128. For example, the event logging module 120 may cause one or more of the glucose measurements that correspond to a time associated with an event representation to be visually emphasized, e.g., the plotted glucose measurement(s) may be enlarged relative to measurements not associated with an event, have a different color or different colors than measurements not associated with an event, have a different shape than measurements not associated with an event, and so forth. Alternatively or additionally, the event logging module 120 may add a visual marker in line with the plotted glucose measurements at the time associated with the respective event representation. By “inline” it is meant that the visual marker may be positioned at the time associated with the event (x-value) and/or approximately at a glucose value (y-value) interpolated from an immediately preceding and an immediately subsequent glucose measurement, e.g., when the time associated with the event is between times of two glucose measurements. These visual markers may be emphasized in manners similar to those discussed just above for emphasizing glucose measurements.

The illustrated example 500 also includes an expanded view 506 of the first event representation 502 and an expanded view 508 of the second event representation 504. The expanded views 506, 508 are included to discuss example details of the first and second event representations 502, 504 as they may be displayed on the glucose graph 128. As depicted in the expanded view 506, the first event representation 502 includes an image 510 of the logged event. In accordance with the described techniques, the event logging module 120 may associate an image with an event in various ways, such as those discussed in more detail above.

In the expanded view 506, the first event representation 502 also includes visual indication 512. The visual indication 512 indicates a characteristic of the event. When different types of events are represented on the glucose graph 128, the visual indications may represent different characteristics depending upon the respective type of event with which the visual indications are displayed. In connection with an event representation that corresponds to a meal, for instance, the visual indication 512 may indicate a classification of the corresponding meal as having generally ‘good’, ‘bad’, or ‘neutral’ healthfulness. In contrast, with an event representation that corresponds to an activity, the visual indication 512 may indicate a classification of how the user feels during the activity, such as whether the user feels generally ‘good’, ‘bad’, or ‘neutral’ during the activity. The visual indication 512 may convey information about various characteristics of an event in the spirit or scope of the described techniques. Additionally, an event representation may include multiple visual indications (e.g., multiple rings) to visually indicate multiple characteristics about an event.

To visually indicate a characteristic, the visual indication 512 may have different visual properties depending on different characteristics. For instance, different colors of the visual indication 512 may be indicative of different characteristics. Given the above example in which an event corresponds to a meal and in which meals may be associated with a ‘good’, ‘bad’, or ‘neutral’ healthfulness, the visual indication 512 may have a first color if it is associated with ‘good’ healthfulness (e.g., green), a second color if it is associated with ‘bad’ healthfulness (e.g., red), or a third color if it is associated with ‘neutral’ healthfulness (e.g., yellow). In this example 500, the visual indication 512 is illustrated as a ring (e.g., a colored ring) surrounding the first event representation 502. A visual indication of an event representation may be configured in a variety of ways to convey information (e.g., a characteristic) about a respective event. As mentioned above, an event representation may also include multiple visual indications.

As depicted in the expanded view 508, the second event representation 504 includes an image 514 of the logged event and a visual indication 516 surrounding the second event representation 504. The image 514 and the visual indication 516 may be configured in a similar manner to the image 510 and the visual indication 512 as discussed in relation to the expanded view 506 of the first event representation 502. By displaying event representations for different types of events on the glucose graph 128 and at positions that correspond to times of the events, the user interface 124 visually conveys effects of different events (e.g., meals, activities, and so forth) on a user's glucose. This visual association between events and glucose measurements can educate a user about which events help the user meet his or her health goals (e.g., help keep the user's glucose measurements within a target range) and which events may be preventing the user from meeting his or her health goals (e.g., cause the user's glucose measurements to be outside the target range).

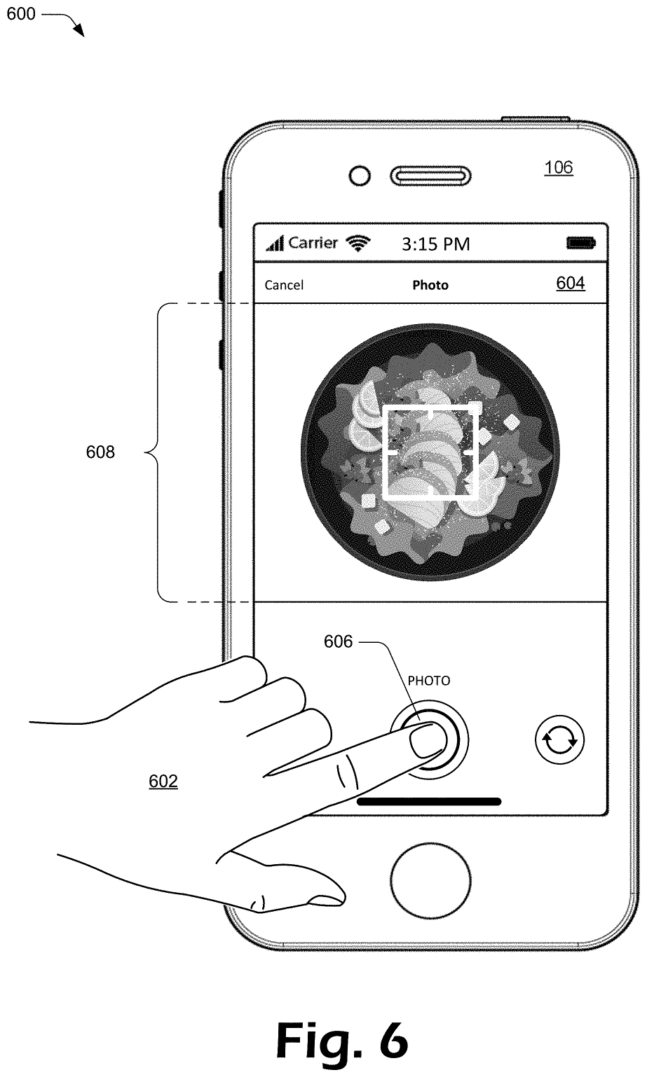

In this example 500, the user interface 124 also includes event logging control 518, which is selectable to log an event and display an event representation on the glucose graph 128 at a position corresponding to a time of the logged event. Although illustrated as a selectable user interface element that is displayed, the event logging control 518 may be configured in different ways to enable an event to be logged and an event representation to be displayed on the glucose graph 128 at a position corresponding to the time of the logged event, such as those discussed in more detail above. Consider now the following discussion of FIGS. 6-8, which describe an example scenario where a meal is logged and a meal representation is displayed on a glucose graph.

FIG. 6 depicts an example 600 of a user interface for capturing an image of a meal to use with a logged meal. In particular, the example 600 includes the computing device 106 from FIG. 1 and a hand 602 of a user, e.g., of the person 102.

In this example 600, the computing device 106 is depicted displaying user interface 604 via a display screen. The user interface 604 includes a selectable instrumentality 606 that is selectable to capture an image (e.g., of a meal) and also includes an image review region 608. In one or more implementations, the image review region 608 is configured to display a livestream of a scene being captured by a camera of the computing device 106 and is displayed responsive to user input to select the selectable instrumentality 606. The livestream may thus act as a preview of an image that may be captured and persisted responsive to selection of the selectable instrumentality 606. Once an image is captured and persisted, responsive to selection of the selectable instrumentality 606, the image may be displayed via the image review region 608. This image may be associated with a meal as described above and below and, in one or more implementations, the image may be incorporated into a meal representation for display. Moreover, the captured and persisted image may be logged and comprise at least a portion of meal data forming a logged meal, which may be stored in the event logs 122. Notably, the meal can be logged automatically without any additional user input from the user other than the user input to the selectable instrumentality 606 to cause the camera to capture the photo of the meal. Thus, responsive to this user input, the event logging module 120 may automatically associate the captured image with the logged meal and incorporate the captured image with the meal representation.

It is to be appreciated that images may be captured and used in connection with logging a variety of other events in addition to meals, e.g., activities, sleep, stress, health events, and so on, in accordance with the described techniques. By way of example, the user may provide input to the selectable instrumentality 606 in order to capture an image of an activity such as a trip to the gym, a soccer match, a trip to a theme park (which may correspond to more walking than usual), an airplane trip (which may include more sitting than usual), and so forth. The ability to quickly and easily log such events by capturing an image enables the user to remember different days, particularly when a day may diverge from the user's typical routine. The user can also capture images in order to log events that may cause stress, such as a wedding day, a funeral, a presentation at work, and so forth. Indeed, the ability to log events by capturing images of a variety of different types events and then providing these images in conjunction with a user's glucose graph enables users to understand how these various events impact their glucose levels.

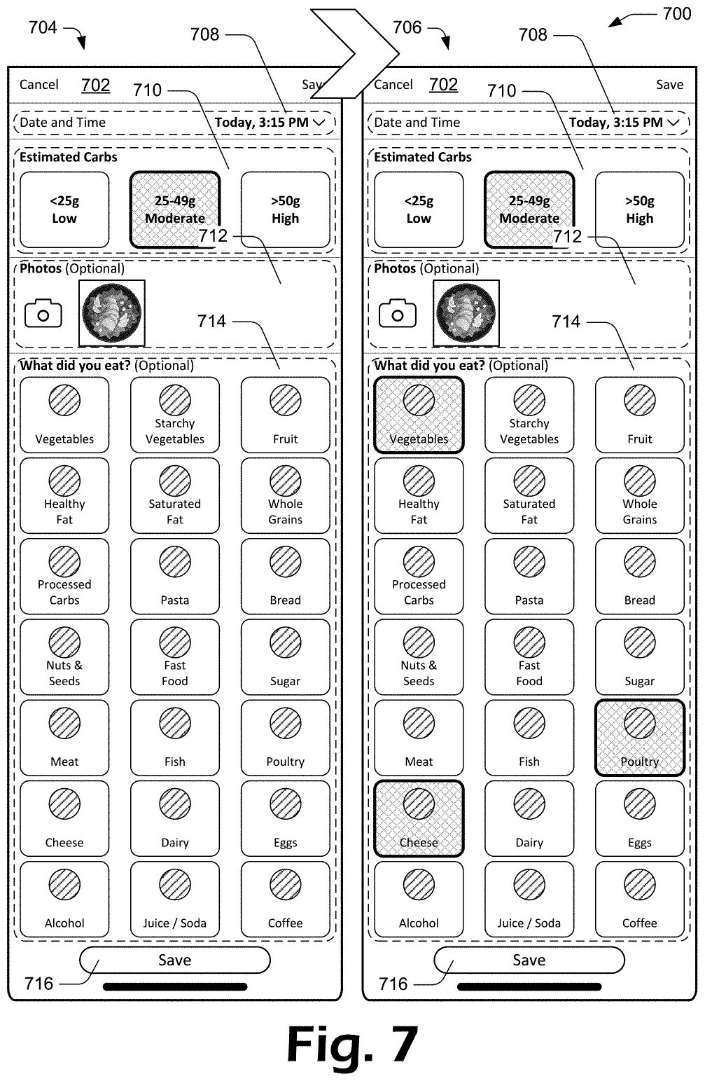

FIG. 7 depicts an example 700 of a user interface which includes graphical elements that are user selectable to provide meal details for logging a meal. In particular, the illustrated example 700 depicts user interface 702 at a first stage 704 and a second stage 706, which is subsequent in time to the first stage 704.

Here, the user interface 702 is depicted including graphical elements that are selectable (e.g., using touch screen functionality) to specify details about a meal to be logged. Selection of such graphical elements corresponds to user input to log a meal in accordance with the described techniques. In one or more implementations, the glucose monitoring application can make it easy for the user to log the “same” or “similar” personalized foods that the user often consumes, such as by display of user-selectable instrumentality in the user interface to log “yesterday's dinner” for “lunch today.” As noted above, details about a meal (or event generally) may be specified in other ways, such as by voice or by determinations made automatically by the event logging module 120.

User interfaces used in connection with the described techniques to receive event data may enable users to provide input to specify a variety of details about an event (e.g., a meal, an activity, or another type of event). As mentioned above, for instance, graphical elements of a logging user interface may enable a user to specify a time of an event or to modify a time automatically determined for the event by the event logging module 120. Additionally or alternatively, graphical elements of such user interfaces may enable a user to specify or otherwise provide other details, such as a title of an event, a type of event (e.g., a meal, an activity, sleep, medication consumption, health event, and so forth), one or more images (e.g., digital photographs) to associate with the event, an icon to associate with the event, a duration of the event, a classification indicative of how the user generally felt during and/or after the event (e.g., ‘good’, ‘fine’, or ‘bad’), a classification of an intensity of an activity (e.g., ‘high’, ‘medium’, or ‘low’), a classification of perceived healthfulness of a meal (e.g., ‘good’, ‘neutral’, or ‘bad’), a classification of a carbohydrate load of a meal (e.g., ‘high’, ‘medium’, or ‘low’), selection of one or more foods eaten as part of a meal, and quality of sleep (e.g., ‘good’, ‘neutral’, or ‘bad’), to name just a few. It is to be appreciated that a variety of details about events, including but not limited to the details mentioned above, may be provided via user interfaces in accordance with the described techniques, and also that various interfaces may be leveraged to receive such details in accordance with the described techniques, such as graphical user interfaces and voice-based interfaces, to name a couple.

Regardless, the illustrated example 700 depicts an implementation in which event details (e.g., meal details) may be specified via a graphical user interface, namely, the user interface 702. In this example 700, the user interface 702 includes date and time portion 708, meal classification portion 710, image association portion 712, food selection portion 714, and commit to log control 716. The date and time portion 708 may be configured to present a date and time associated with a meal being logged. In one or more implementations, the date and time portion 708 may include one or more user interface elements in relation to which a user may provide input to specify and/or modify the date and time associated with the meal being logged.

The meal classification portion 710 may include one or more graphical elements in relation to which a user may provide input to classify a meal being logged. In the illustrated example 700, for instance, the user interface 702 includes graphical elements that are selectable to classify the meal being logged as a low, moderate, or high carbohydrate meal. In accordance with the described techniques, the classification selected may correspond to a characteristic of the meal. As such, this classification may be used as a basis for configuring a visual indication of a meal representation for the meal being logged. In this example 700, the ‘Moderate’ graphical element in the meal classification portion 710 is depicted having been selected. By way of example, this element may have been selected by a user interacting with the user interface 702 or by the event logging module 120, such as based on an analysis performed by the event logging module 120 of an image of the meal.

The image association portion 712 may include one or more graphical elements in relation to which a user may provide input to associate an image (e.g., a photograph) of a meal being logged. In the illustrated example 700, the image association portion 712 includes a thumbnail of an image. Responsive to selection of the commit to log control 716 this image may be associated with the meal being logged. In one or more implementations, such images may be captured based on user input received via the user interface 604. Additionally, such images may be used for configuring an image portion of a meal representation for the meal being logged.

The food selection portion 714 may include one or more graphical elements in relation to which a user may provide input to specify foods consumed as part of the meal being logged. In this example 700, there are a plurality of elements which each indicate a different food. In one or more implementations, multiple such elements may be selectable, e.g., to specify multiple foods being consumed in connection with the meal being logged. In other implementations, the system may limit a user to selecting only a single element to specify a single identified food being eaten for the meal being logged. It is to be appreciated that the depicted elements are merely examples and in implementation a user interface may enable users to specify fewer, additional, and/or different foods without departing from the spirit or the scope of the described techniques. Additionally or alternatively, icons of selected graphical elements in the food selection portion 714 may be used for configuring an image of a meal representation for the meal being logged, e.g., when a photograph of the meal is not captured.

In contrast to the first stage 704, the second stage 706 depicts graphical elements in the food selection portion 714 having been selected, e.g., the ‘Vegetables’, ‘Poultry’, and ‘Cheese’ elements. These elements may have been selected by a user interacting with the user interface 702 or by the event logging module 120, such as based on an analysis performed by the event logging module 120 of an image of the meal.

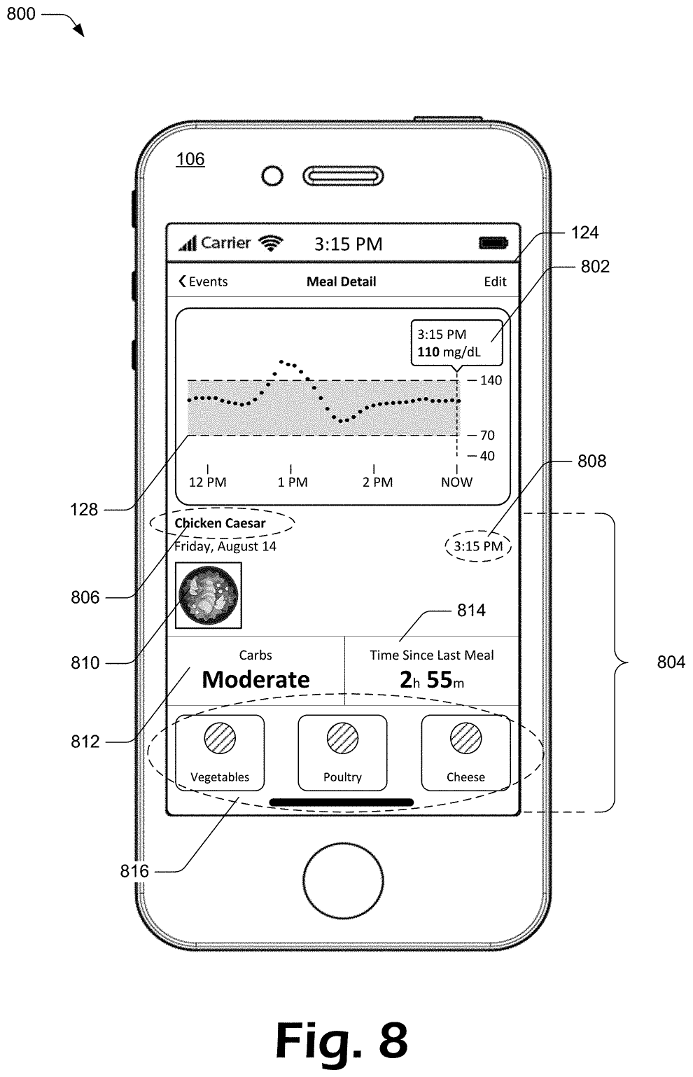

In one or more implementations, the commit to log control 716 may be selectable to log the respective meal, e.g., save the meal to one or more of the event logs 122. Responsive to selection of the commit to log control 716, for instance, the event logging module 120 may log the meal for which the details are provided via the user interface 702. Further, the event logging module 120 may cause a meal representation of the meal to be generated and displayed on a glucose graph, e.g., at a position that corresponds to a time associated with the meal. In the context of displaying the meal representation on the glucose graph, consider the following further example of FIG. 8.

FIG. 8 depicts an example 800 of a user interface displaying meal logging visual representations as text information with glucose measurements.

The illustrated example 800 includes from FIG. 1 an example of the computing device 106 displaying an example of the user interface 124 via a display device, e.g., a touchscreen. Here, the user interface 124 includes the glucose graph 128, which plots one or more of the glucose measurements 114 (e.g., of the person 102) over time. In the illustrated example 300, the time period over which the glucose measurement 114 are displayed in the glucose graph 128 spans a plurality of hours up to a current time—indicated by the time label ‘NOW’. As mentioned above, the glucose monitoring application 116 may plot the glucose measurements 114 on the glucose graph 128 over different periods of time without departing from the spirit or scope of the techniques described herein, and the depicted time period is merely another example.

In contrast to the meal representations discussed above, the illustrated example 800 includes a text-based meal representation 802 for the respective logged meal. In other words, rather than being configured with an image representative of the respective meal, the text-based meal representation 802 includes text information associated with a meal. Here, the text-based meal representation 802 includes the text information displayed in a graphical element (e.g., a bubble) on the glucose graph 128. The text-based meal representation 802 may include various information in accordance with the described techniques. In this example 800, the text-based meal representation 802 includes a time of the meal and a glucose measurement at the time. Alternatively or additionally, a text-based meal representation may include any one or more of a meal name (or title), a description of the meal, one or more foods of the meal, a carbohydrate load of the meal, a general healthfulness classification, a classification of how the user feels at the time of eating the meal and/or after, a location where the meal is consumed (e.g., restaurant), and so forth. It is to be appreciated that these examples are provided by way of example and not limitation and that a text-based meal representation may include different information in accordance with the described techniques.

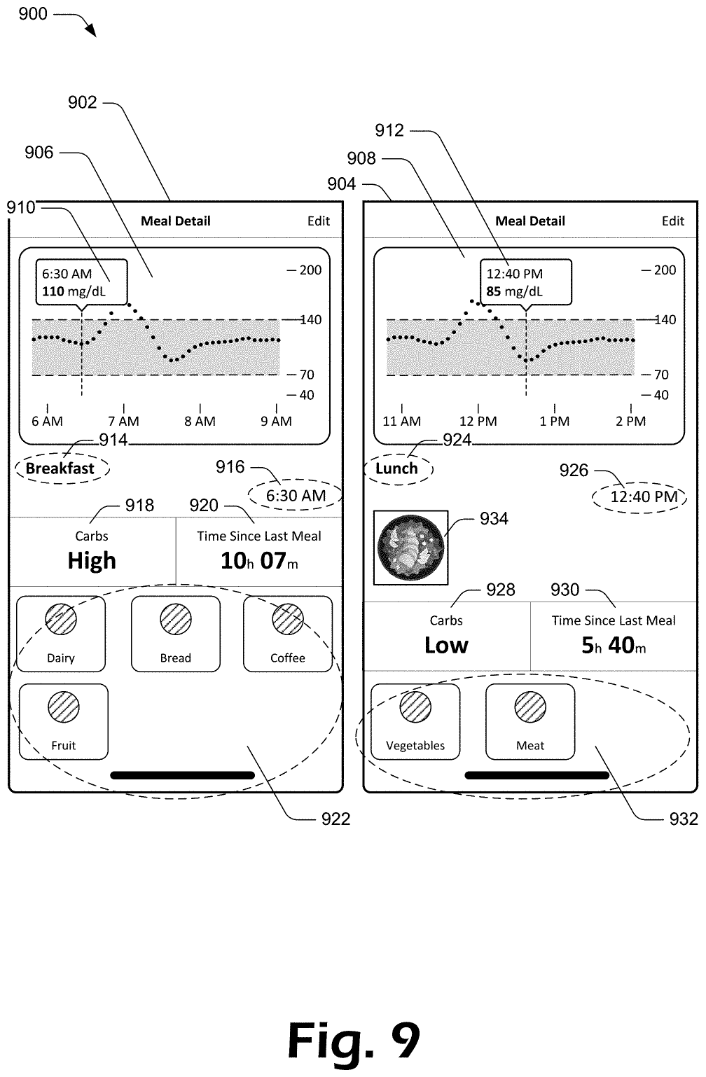

In this example 800, the user interface 124 also includes supplemental information 804. Here, the supplemental information includes a meal title 806, a meal time 808, a meal image 810, an estimated carbohydrate load of the meal 812, an amount of time since a last meal 814, and foods included in the logged meal 816. As noted above, the illustrated example 800 is a continuation of the scenario discussed in relation to FIGS. 6 and 7. By way of example, the user interface 124 as displayed in the example 800 may be displayed after details about a meal have been specified, e.g., after a user selects the commit to log control 716 of the user interface 702. To this end, the supplemental information 804 is consistent with the depicted selections of the illustrated example 700. For instance, the meal time 808 matches the time indicated in the date and time portion 708 of the user interface 702 (e.g., 3:15 PM), the estimated carbohydrate load of the meal 812 matches the element depicted selected in the meal classification portion 710 (e.g., Moderate), the meal image 810 matches the image depicted in the image association portion 712, and the foods included in the logged meal 816 match the elements depicted selected in the food selection portion 714 at the second stage 706 (e.g., ‘Vegetables’, ‘Poultry’, and ‘Cheese’). Supplemental information displayed via user interfaces having event representations presented on glucose graphs may describe a variety of details about a meal without departing from the spirit or scope of the described techniques. Moreover, the user interface 124 may be configured in a variety of ways and include different information (e.g., depending on what information, if any, a user specifies via user input and/or depending on an event type) in accordance with the described techniques. In this context, consider the following discussion of FIG. 9, which depicts different interface configurations for a logged meal without an associated image and a different logged meal with an associated image.

FIG. 9 depicts further examples 900 of user interfaces displaying meal logging visual representations as text information with glucose measurements.

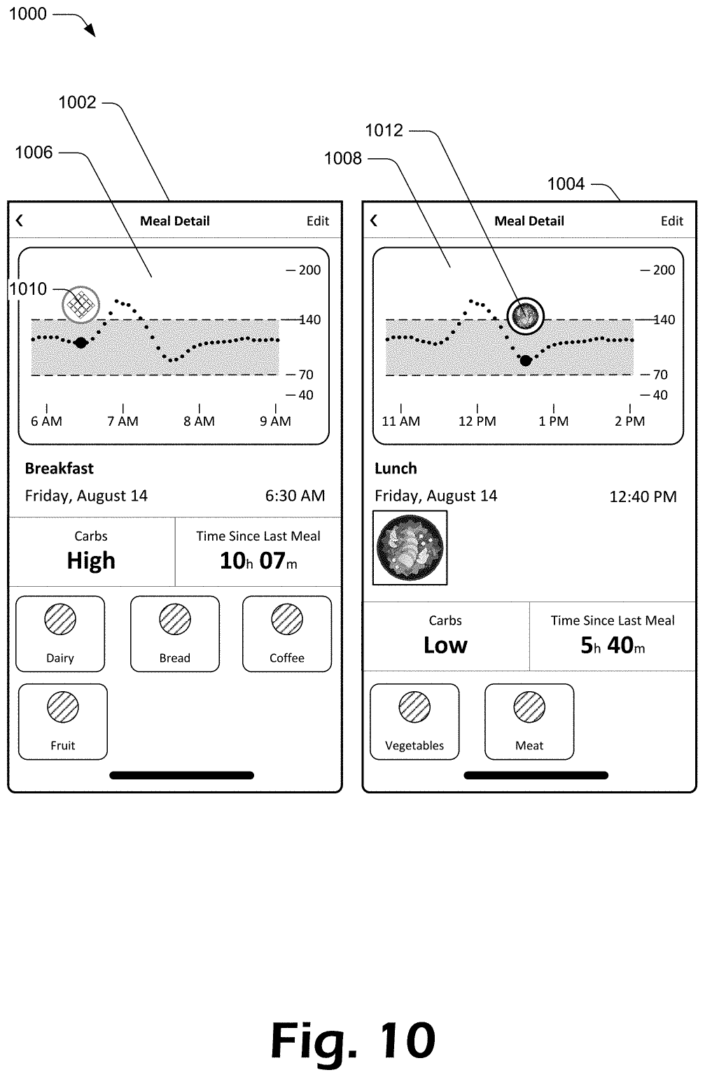

In particular, the illustrated example 900 includes a first user interface 902 and a second user interface 904 to demonstrate different example configurations of graphical elements that may be displayed in connection meal representations on a glucose graph. Here, the first and second user interfaces 902, 904 both include respective glucose graphs 906, 908. The first and second user interfaces 902, 904 also both include text-based meal representations 910, 912. However, the first and second user interfaces 902, 904 are depicted presenting slightly different supplemental information. For example, the first user interface 902 includes meal title 914, meal time 916, estimated carbohydrate load of the meal 918, amount of time since a last meal 920, and foods included in the logged meal 922, but does not include a meal image. The second user interface 904 includes meal title 924, meal time 926, estimated carbohydrate load of the meal 928, amount of time since a last meal 930, and foods included in the logged meal 932, but in contrast to the first user interface 902 also includes a meal image 934. As further examples of how the user interfaces displayed in connection with the described techniques may differ, consider also the following discussion of FIG. 10.

FIG. 10 depicts further examples 1000 of user interfaces displaying meal logging visual representations as meal images or icons with glucose measurements.

The illustrated example 1000 includes a first user interface 1002 and a second user interface 1004 to demonstrate different examples of the meal representations that may be displayed in connection with the described techniques. The first and second user interfaces 1002, 1004 are the same as the first and second user interfaces 902, 904, except that the first and second user interface 1002, 1004 present non-text based meal representations on glucose graphs rather than text-based representations. In this example 1000, these non-text based meal representations are configured in a similar manner as those discussed in relation to FIGS. 3-5.

Like the first and second user interfaces 902, 904 of the example 900, the first and second user interfaces 1002, 1004 include respective glucose graphs 1006, 1008. In contrast to the first and second user interfaces 902, 904 of the example 900, though, the first and second user interfaces 1002, 1004 of this example 1000 each include a respective meal representation 1010, 1012 having an image or icon and a visual indication as in the examples depicted in FIGS. 3-5. The first and second user interfaces 1002, 1004 also include supplemental information that is similar to the supplemental information of the first and second user interfaces 902, 904.

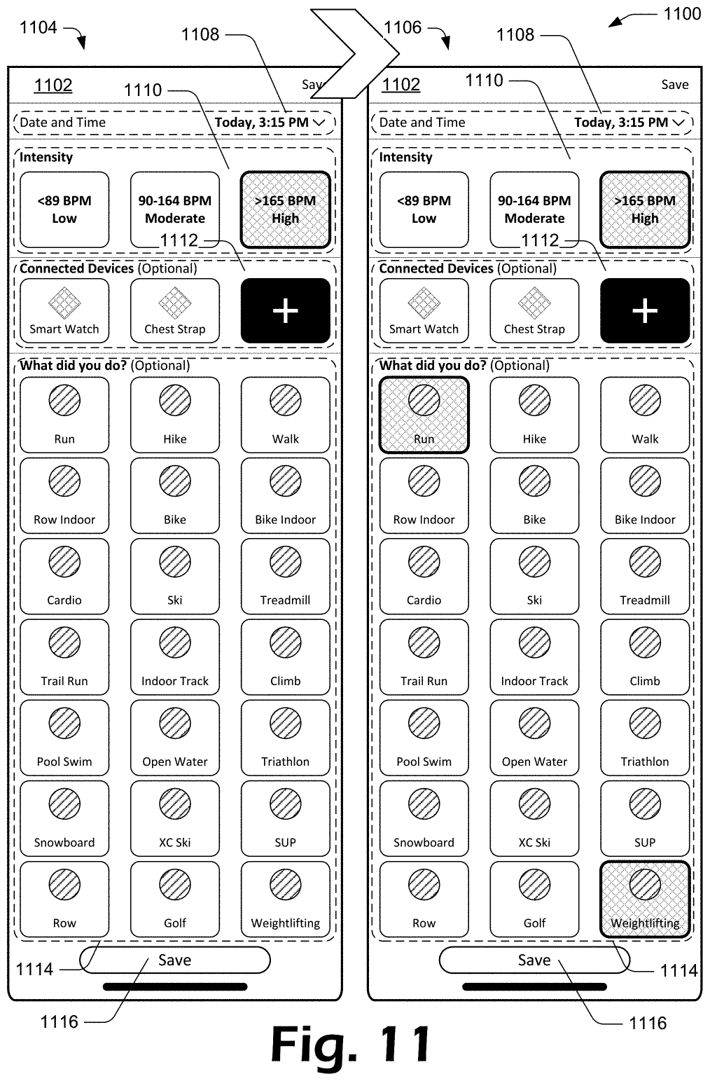

FIG. 11 depicts an example 1100 of a user interface which includes graphical elements that are user selectable to provide activity details for logging an activity. In particular, the illustrated example 1100 depicts user interface 1102 at a first stage 1104 and a second stage 1106, which is subsequent in time to the first stage 1104.

Here, the user interface 1102 is depicted including graphical elements that are selectable (e.g., using touch screen functionality) to specify details about an activity to be logged. Selection of such graphical elements corresponds to user input to log an activity in accordance with the described techniques. As noted above, details about an activity (or event generally) may be specified in other ways, such as by voice or by determinations made automatically by the event logging module 120.

Regardless of the various ways in which details about events such as activities may be provided, the illustrated example 1100 depicts an implementation in which event details (e.g., activity details) may be specified via a graphical user interface, namely, the user interface 1102. In this example 1100, the user interface 1102 includes date and time portion 1108, activity classification portion 1110, health tracking device association portion 1112, activity selection portion 1114, and commit to log control 1116. The date and time portion 1108 may be configured to present a date and time associated with an activity being logged. In one or more implementations, the date and time portion 1108 may include one or more user interface elements in relation to which a user may provide input to specify and/or modify the date and time associated with the activity being logged.

The activity classification portion 1110 may include one or more graphical elements in relation to which a user may provide input to classify an activity being logged. In the illustrated example 1100, for instance, the user interface 702 includes graphical elements that are selectable to classify the activity being logged as a low, moderate, or high intensity. In accordance with the described techniques, the classification selected may correspond to a characteristic of the activity. As such, this classification may be used as a basis for configuring a visual indication of an activity representation for the activity being logged. In this example 1100, the ‘High’ graphical element in the activity classification portion 1110 is depicted having been selected. By way of example, this element may have been selected by a user interacting with the user interface 1102 or by the event logging module 120, such as based on an analysis performed by the event logging module 120 of data obtained from a connected health tracking device or application or data produced by one or more sensors (e.g., accelerometer) of the computing device 106.

The health tracking device association portion 1112 may be configured to present graphical elements indicating health tracking devices or applications from which activity data may be obtained by the event logging module 120 for logging an activity. The event logging module 120 may obtain a variety of activity data from health tracking devices and/or applications to incorporate into a logged activity. By way of example and not limitation, examples of the activity data that may be obtained include activity intensity, activity duration, calories burned, activities performed, heart rate, heart rate variability (HRV), VO₂ max (e.g., maximal oxygen consumption), an amount of work performed by the user during the activity, power output during the activity, a score of the user in connection with the activity (e.g., a golf score), one or more distances associated with the activity (e.g., a distance traversed or amount of vertical gained), and so forth. It is to be appreciated that a variety of other activity data may also or alternatively be obtained from connected health tracking devices or applications without departing from the spirit or scope of the described techniques.

Graphical elements of the health tracking device association portion 1112 may be selectable to remove a health tracking device or application (e.g., so that data produced in connection with the device is not used in connection with activity logging), to configure settings of a health tracking device or application, and so forth. At least one graphical element of the health tracking device association portion 1112 may be selectable to add a health tracking device or application, i.e., so that data produced in connection with the device or application is used in connection with activity logging.

The activity selection portion 1114 may include one or more graphical elements in relation to which a user may provide input to specify active components performed and/or devices used as part of the activity logged. In this example 1100, there are a plurality of elements which each indicate a different active component. In one or more implementations, multiple such elements may be selectable, e.g., to specify multiple active components being performed in connection with the activity being logged. In other implementations, the system may limit a user to selecting only a single element to specify a single active element being performed for the activity being logged. It is to be appreciated that the depicted elements are merely examples and in implementation a user interface may enable users to specify fewer, additional, and/or different active elements without departing from the spirit or the scope of the described techniques. Additionally or alternatively, icons of selected graphical elements in the activity selection portion 1114 may be used for configuring an image of an activity representation for the activity being logged, e.g., when a photograph associated with the activity is not captured.

In contrast to the first stage 1104, the second stage 1106 depicts graphical elements in the activity selection portion 1114 having been selected, e.g., the ‘Run’ and ‘Weightlifting’ elements. These elements may have been selected by a user interacting with the user interface 1102 or by the event logging module 120, such as based on an analysis performed by the event logging module 120 of activity data obtained from a connected health tracking device or application.

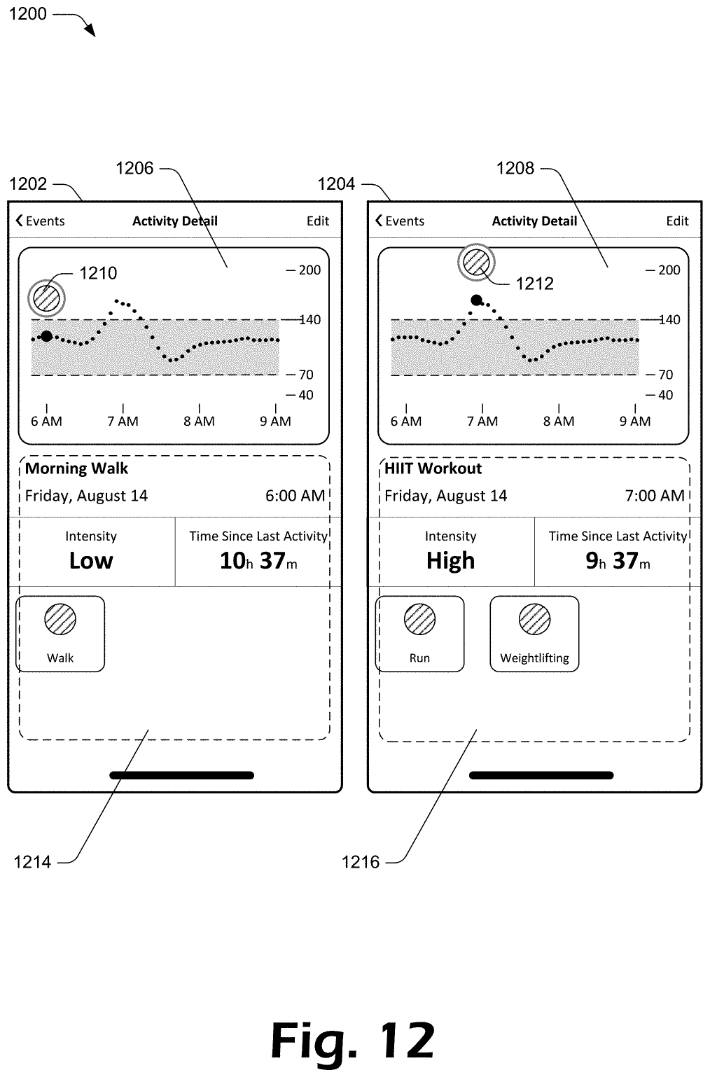

In one or more implementations, the commit to log control 1116 may be selectable to log the respective activity, e.g., save the activity to one or more of the event logs 122. Responsive to selection of the commit to log control 1116, for instance, the event logging module 120 may log the activity for which the details are provided via the user interface 1102. Further, the event logging module 120 may cause an activity representation of the activity to be generated and displayed on a glucose graph, e.g., at a position that corresponds to a time associated with the activity. In the context of displaying the activity representation on the glucose graph, consider the following further examples of FIG. 12.

FIG. 12 depicts further examples 1200 of user interfaces displaying activity logging visual representations as activity images or icons with glucose measurements.

In particular, the illustrated example 1200 includes a first user interface 1202 and a second user interface 1204. Here, the first and second user interface 1202, 1204 both include respective glucose graphs 1206, 1208. The first and second user interfaces 1202, 1204 also both include activity representations 1210, 1212. Here, the first and second user interfaces 1202, 1204 each present only a single activity representation. This may correspond to a scenario in which a user selects to view details of a single activity. Although a single activity representation is depicted for each of the first and second user interfaces 1202, 1204 in the illustrated example 1200, it is to be appreciated that in one or more implementations a user interface may display multiple activity representations (or event representations) in accordance with the described techniques, examples of which are depicted in FIGS. 3-5.

In this example 1200, the first and second user interfaces 1202, 1204 are also depicted presenting respective supplemental information 1214, 1216 about the respective logged activity. In particular, the first and second user interfaces 1202, 1204 are depicted presenting activity titles, activity times, activity intensities, amounts of time since last activities, and selected active elements.

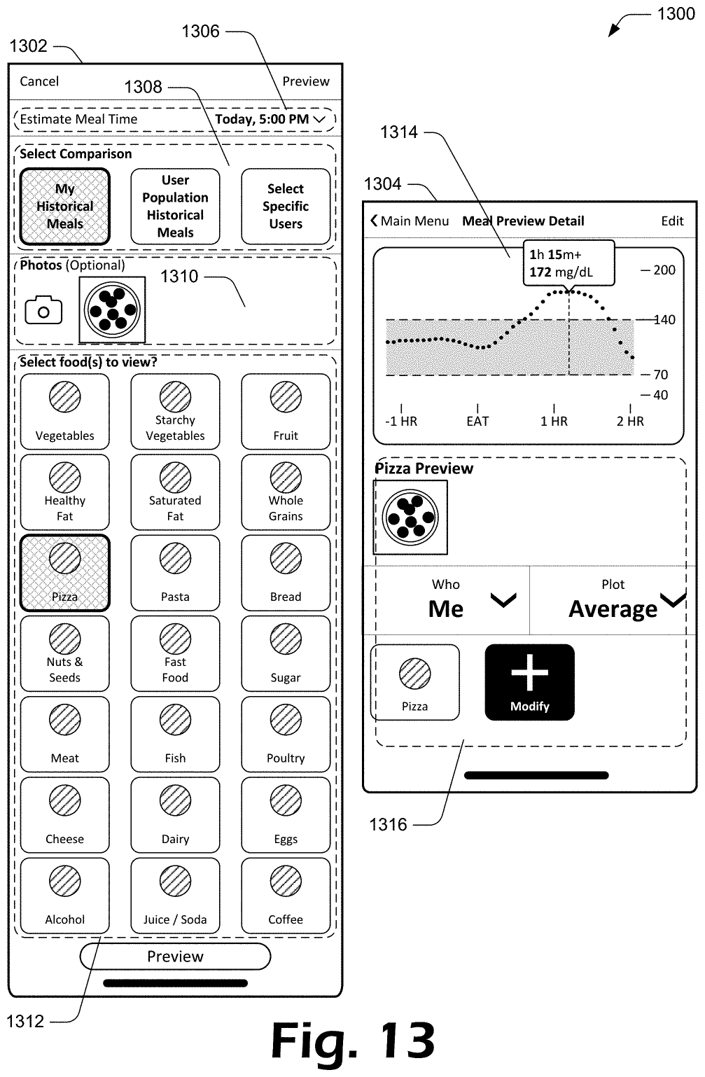

FIG. 13 depicts an example 1300 of user interfaces used in connection with selecting a meal to view its historical effects on glucose measurements.

In one or more implementations, the glucose monitoring application 116 is configured to display one or more user interfaces to visually convey how selected foods have historically affected glucose of a user of the glucose monitoring application 116 (e.g., the person 102) and/or how the selected foods have historically affected the glucose of one or more users of the user population 108. By way of example, the glucose monitoring application 116 may present user interface elements (e.g., menu options) that enable a user to select a food for which the user would like to view how his or her glucose is affected by at least one selected food. The user may also select a variety of other options in connection with such a selection, such as to view overlays of historical glucose measurements for multiple instances in which the user has eaten the selected food and/or to view average glucose measurements determined from multiple such instances in which the user has eaten the selected food. Additionally or alternatively, the user interfaces may include elements that are selectable to display the user's historical glucose measurements when the selected food has been eaten concurrently with historical glucose measurements of the user population 108 which correspond to having eaten the selected food. By presenting this information, the glucose monitoring application 116 visually conveys to a user how consuming particular foods has affected the user historically. Further still, by presenting this information the glucose monitoring application 116 can visually convey to a user how consuming particular foods has affected the user historically in relation to how other users are affected by consuming the particular food.

As one example implementation of user interfaces that support such functionality, consider the following discussion of first and second user interfaces 1302, 1304. Broadly speaking, the first user interface 1302 is an example of a user interface with which a user may interact to select a meal and/or specify details about the meal for which the user would like to be presented historical glucose information, e.g., so that the user can see how the meal or similar meals have affected his or her glucose. Here, the first user interface 1302 includes estimated meal time portion 1306, a historical data to view portion 1308, a meal image portion 1310, and a food selection portion 1312.

Generally speaking, the estimated meal time portion 1306 may be configured to present a time at which a user estimates he or she will consume a meal. In one or more implementations, the estimated meal time portion 1306 may include one or more user interface elements in relation to which a user may provide input to specify and/or modify the time for the meal. The historical data to view portion 1308 may include one or more graphical elements that are selectable to specify the glucose measurements presented via the second user interface 1304, e.g., whether only glucose measurements of the user (e.g., the person 102) are presented, whether only glucose measurements of the user population 108 are presented, whether glucose measurements of the user and one or more other users of the user population 108 are presented, and so forth. The illustrated example 1300 represents a scenario where a user has selected to view only his or her own glucose measurements via the second user interface 1304—not glucose measurements of other users of the user population 108.

The meal image portion 1310 may include one or more graphical elements in relation to which a user may provide input to associate an image of a meal. In such scenarios, the event logging module 120 may analyze an associated image to identify the food for which the display of historical glucose measurements is presented (e.g., whether the food is a hot dog or whether the food is pizza) and/or to determine a nutritional makeup of the meal. The food selection portion 1312 may include one or more graphical elements in relation to which a user may provide input to specify foods for which he or she would like to be presented historical glucose measurements. Although a single food is depicted selected in the illustrated example 1300, in operation a user may select multiple foods to be shown how a combination of the multiple selected foods has historically affected his or her glucose (or affected the glucose of other users).

The second user interface 1304 includes glucose graph 1314, which includes historical glucose measurements 114 of the user (e.g., the person 102) that correspond to the selected meal. In this particular example 1300, the historical glucose measurements plotted on the glucose graph 1314 correspond to average glucose measurements when the user has eaten the selected food. In one or more implementations, the glucose graph 1314 may plot actual glucose measurements for multiple times that the user has eaten the selected food, e.g., multiple glucose traces that correspond to eating the selected food may be overlaid. Here, the historical glucose measurements plotted on the glucose graph 1314 are plotted in relation to a time the selected food was eaten and relative times before and after the meal, e.g., (−1 HR=1 hour before the selected meal is consumed). The second user interface 1304 is also depicted with supplemental information 1316 about the preview, which visually informs the user whose historical glucose is presented as well as the food for which the historical glucose is being presented. In one or more implementations, the user interface may also include functionality which enables the user to explore the context of a particular meal (e.g., timing of the meal and activities performed in relation to the meal) in order to determine whether the context of a particular meal changes the impact of the meal on the user's glucose. For example, based on this functionality, the user may learn that with a certain context a particular meal historically had a positive or neutral effect on their glucose (e.g., glucose within the target range after the meal), whereas with a different context the same meal historically had a negative effect on their glucose (e.g., out of target range after meal). For example, the user can explore the context associated with the meal of “pizza” in order to determine that consuming pizza after 8 pm with no walk afterwards has a negative effect on glucose, whereas consumption of pizza at 12 pm followed by a 30 minute walk afterwards keeps the user's glucose within the target range

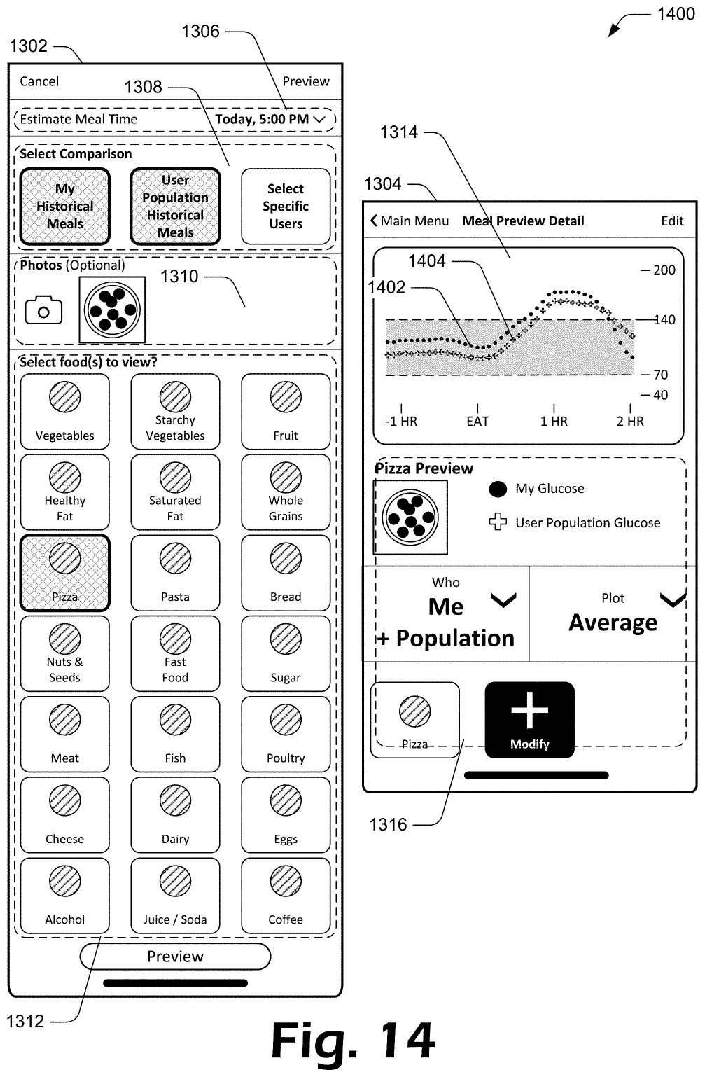

FIG. 14 depicts a further example 1400 of user interfaces used in connection with selecting a meal to view its historical effects on glucose measurements, including historical effects on glucose measurements of a population of users.

This example 1400 includes the first and second user interfaces 1302, 1304 from the example 1300 discussed just above. In contrast to the example 1300, however, the illustrated example 1400 represents a scenario where a user has selected to view his or her own historical glucose measurements via the second user interface 1304 along with historical glucose measurements of the user population 108—not only his or her own glucose measurements. This selection is indicated in the illustrated example 1400 by the visual emphasis on two of the graphical elements depicted in the historical data to view portion 1308. In contrast to the example 1300 where only the ‘My Historical Meals’ element is visually emphasized (indicating selection) in the historical data to view portion 1308, in the example 1400 both the ‘My Historical Meals’ element and the ‘User Population Historical Meals’ elements are visually emphasized (indicating selection of both). Accordingly, the second user interface 1304 is depicted in the example 1400 displaying both historical glucose of the user 1402 and concurrently historical glucose of one or more users 1404 of the user population 108 on the glucose graph 1314. Although the illustrated example 1400 indicates that the displayed measurements are averages, in one or more implementations non-averaged glucose may be displayed on the glucose graph 1314, such as one or more actual traces of the person 102's glucose measurements.

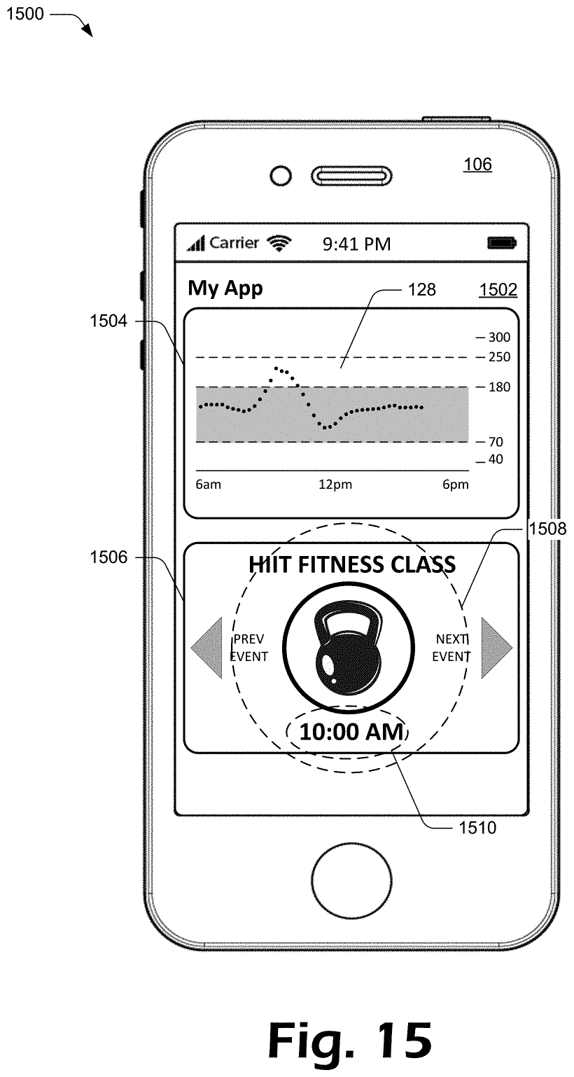

FIG. 15 depicts another example 1500 of a user interface displaying event logging visual representations with glucose measurements. In one or more implementations, event logging visual representations may not be displayed on a glucose graph. Instead, event logging visual representations may be displayed proximate a glucose graph, such by displaying the glucose graph and the event logging visual representations in separate frames, cards, or tiles of a user interface. The relationship between the events being logged may be conveyed in different ways from the previously discussed implementations where the event logging representations are displayed on the glucose graph, such as by instead including a time or a visual indicator representative of the time as part of the proximate display of an event representation.

The illustrated example 1500 includes the computing device 106, which is depicted displaying user interface 1502. Here, the user interface 1502 is shown presenting a first card 1504 and a second card 1506. The first card 1504 includes the glucose graph 128 and the second card 1506 includes an event representation 1508, which represents a logged or otherwise tracked event, e.g., tracked by a wearable fitness device, tracked as a result of checking into a location, tracked as a result of logging on to an online class (a video conference for the class), and so forth.

Although the event representation 1506 is not displayed within the first card 1504 on the glucose graph 128, the event representation 1508 includes a visual indication 1510 of the relationship between the event and the glucose graph 128. Here, the visual indication 1510 corresponds to a time associated with the respective event, however, it is to be appreciated that a card having an event representation may indicate a relationship to the proximate glucose graph 128 in a variety of ways without departing from the spirit or scope of the described techniques. Additionally, a card (or other user interface element) displayed proximate a separate card (or other user interface element) with a glucose graph may display or otherwise present a variety of information in accordance with the described techniques.

This proximate display of glucose graph and event logging visual representations—as it contrasts with display of event logging visual representations on the glucose graph—may be advantages in scenarios where at least one of the user interface 1502, the glucose graph, or the event representation 1508, corresponds to a different platform from the others. In one example, for instance, the user interface 1502 may correspond to a mobile application of a first service provider (e.g., a third-party platform), while the first card 1504 (and the glucose graph 128) and the second card 1506 (and the event representation 1508) correspond to a second service provider (e.g., the glucose monitoring platform 110). In this way, the first card 1504 and the second card 1506 may be considered “guest” displays on the third-party's mobile application. In another example, the user interface 1502 and the second card 1506 may correspond to a first service provider (e.g., the third-party platform) and the first card 1504 may correspond to a second service provider (e.g., the glucose monitoring platform 110), such that the first card 1504 is considered a guest display. In still another example, the user interface 1502 may correspond to a first service provider (e.g., the third-party platform), the first card 1504 may correspond to a second service provider (e.g., the glucose monitoring platform 110), and the second card 1506 may correspond to a third service provider (e.g., a different third-party platform from the first service provider). In this third example, the first card 1504 and the second card 1506 may be considered guest displays on the user interface 1502, but from different “guests”, e.g., the glucose monitoring platform 110 and the different third-party platform, respectively.

It is to be appreciated that the second card 1506 may be configured with various user interface elements for interacting with event representations in one or more implementations, for example, the second card may include user interface elements for navigating (e.g., scrolling) through events (e.g., chronologically). The first card 1504 and the second card 1506 may also be selectable and, responsive to selection, initiate a variety of behaviors, such as launching a corresponding application, displaying more information, and displaying a menu with further selectable actions, to name just a few.

Having discussed exemplary details of the techniques for meal and activity logging with a glucose monitoring user interface, consider now some examples of procedures to illustrate additional aspects of the techniques.

Example Procedures

This section describes examples of procedures for meal activity logging with a glucose monitoring interface. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations the procedures are performed by a glucose monitoring application, such as the glucose monitoring application 116 that makes use of the event logging module 120.

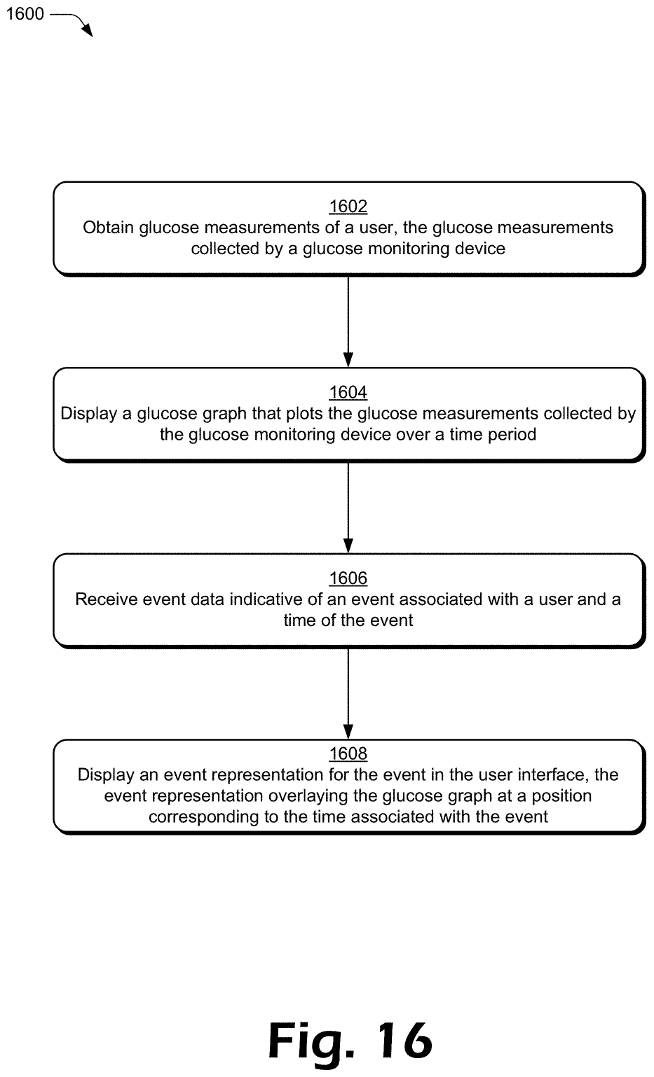

FIG. 16 depicts a procedure 1600 in an example of an implementation in which an event representation of an event is displayed in a user interface with a glucose graph.

Glucose measurements of a user are obtained (block 1602). In accordance with the principles discussed herein, the glucose measurements are collected by a glucose monitoring device. By way of example, the glucose monitoring application 116 implemented at the computing device 106 obtains the glucose measurements 114 of the person 102 from the wearable glucose monitoring device 104.

A user interface is displayed that includes a glucose graph that plots the glucose measurements collected by the glucose monitoring device over a time period (block 1604). By way of example, the glucose monitoring application 116 implemented at the computing device 106 displays, via its display device, a user interface 124 that includes the glucose graph 128, which plots one or more of the glucose measurements 114 (e.g., of the person 102) over time.

Event data indicative of an event associated with a user and a time of the event is received (block 1606). By way of example, the event logging module 120 is configured to enable events (e.g., meals or activities) to be added to the event logs 122, including by receiving data describing or otherwise associated with events. As described throughout, the event may correspond to a meal consumed by the user, or one of various activities performed by the user, such as exercise, meditation, sleep, and so forth. In some cases, the event data is received responsive to user input to log an event, such as via user input to the selectable instrumentality 606 to capture an image (e.g., of a meal) using a camera of the computing device 106, or a spoken voice command to log a meal or an event. The event logging module 120 may also import event data from other applications, such as to import event data from a meal logging application or a health tracking application (or device) that is separate from the glucose monitoring application 116.

An event representation for the event is displayed in the user interface (block 1608). In accordance with the principles discussed herein, the event representation overlays the glucose graph at a position corresponding to the time associated with the event. By way of example, the glucose monitoring application 116 displays one or more event representations 126 on the user interface 124 with a glucose graph 128, e.g., that plots one or more of the glucose measurements 114 over time.

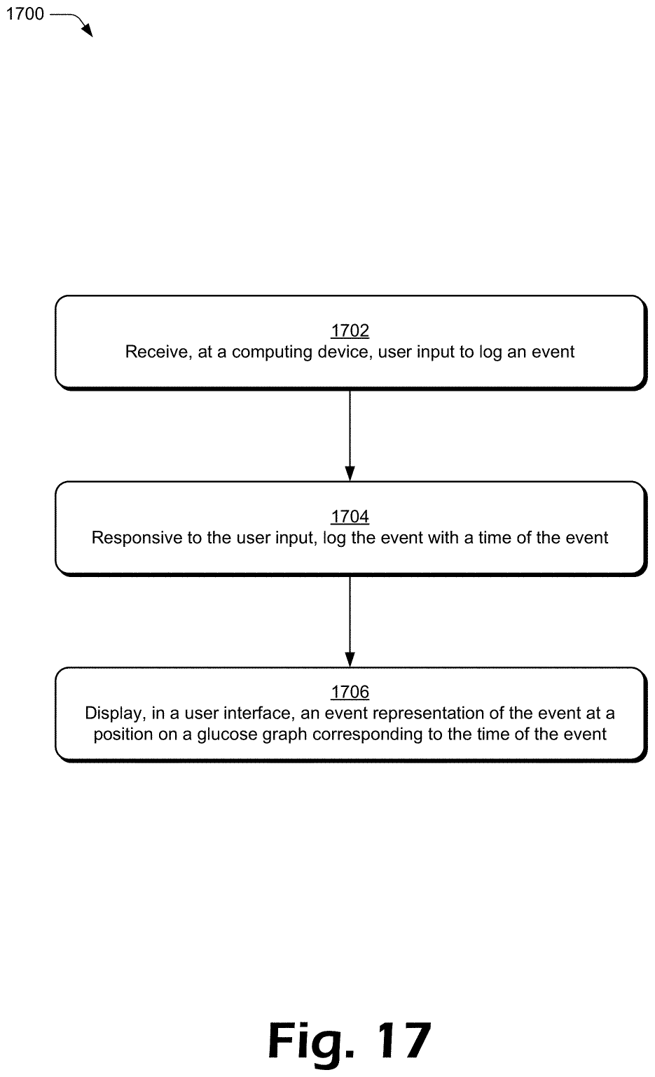

FIG. 17 depicts a procedure 1700 in an example of an implementation in which an event is logged responsive to user input and an event representation of the event is displayed in a user interface along with a glucose graph.

User input is received, at a computing device, to log an event (block 1702). By way of example, the event logging module 120 receives user input to log an event. The user input to log the event may be received in a variety of different ways, such as via user input to the selectable instrumentality 606 to capture an image (e.g., of a meal), or a spoken voice command to log a meal or an event. As described throughout, user input can be received to log a variety of different types of events, including meals consumed by the user, or one of various activities performed by the user, such as exercise, meditation, sleep, and so forth.

Responsive to the user input, the event is logged with a time of the event (block 1704). By way of example, the event logging module 120 logs the event with a time of the event in the event logs 122. As discussed throughout, the event logging module 120 may associate a time with an event by identifying a time (e.g., via a timestamp) when a photo of the event is captured, e.g., using a camera of the computing device 106. Alternatively or additionally, the event logging module 120 may receive a user entry of a time for the event via one or more instrumentalities, e.g., text entry, dropdown box, one or more time scroll wheels, or tapping on the glucose graph to add an event, to name just a few. Indeed, the event logging module 120 may associate times with events in a variety of ways without departing from the spirit or scope of the techniques described herein.

An event representation of the event is displayed, in a user interface, at a position on a glucose graph corresponding to the time of the event (block 1706). By way of example, the glucose monitoring application 116 displays one or more event representations 126 on the user interface 124 with a glucose graph 128, e.g., that plots one or more of the glucose measurements 114 over time. As an example of displaying event representations for a meal, the user interface 124 includes the first and second meal representations 302, 304, which are presented on the glucose graph 128 in the illustrated example 300. Notably, the event logging module 120 causes the first and second meal representations 302, 304 to be presented at positions on the glucose graph 128 that correspond to times associated with the respective meals logged.

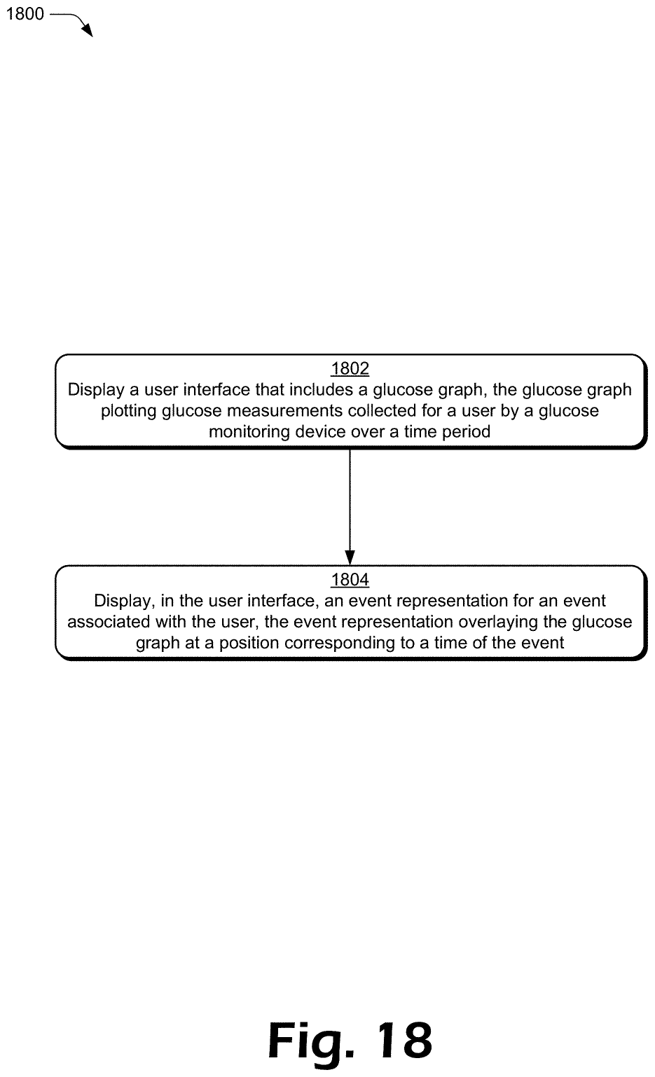

FIG. 18 depicts an additional procedure 1800 in an example of an implementation in which an event representation of an event is displayed in a user interface with a glucose graph.

A user interface that includes a glucose graph is displayed (block 1802). In accordance with the principles discussed herein, the glucose graph plots glucose measurements collected for a user by a glucose monitoring device over a time period. By way of example, the glucose monitoring application 116 implemented at the computing device 106 displays, via its display device, a user interface 124 that includes the glucose graph 128, which plots one or more of the glucose measurements 114 (e.g., of the person 102) over time. The glucose monitoring application 116 obtains the glucose measurements 114 of the person 102 from the wearable glucose monitoring device 104.

An event representation for an event associated with the user is displayed in the user interface (block 1804). In accordance with the principles discussed herein, the event representation overlays the glucose graph at a position corresponding to a time of the event. By way of example, the glucose monitoring application 116 displays one or more event representations 126 on the user interface 124 with a glucose graph 128, e.g., that plots one or more of the glucose measurements 114 over time. As described throughout, the event representations may represent events such as meals consumed by the user, and/or various activities performed by the user, such as exercise, meditation, sleep, and so forth.

Having described examples of procedures in accordance with one or more implementations, consider now an example of a system and device that can be utilized to implement the various techniques described herein.

Example System and Device

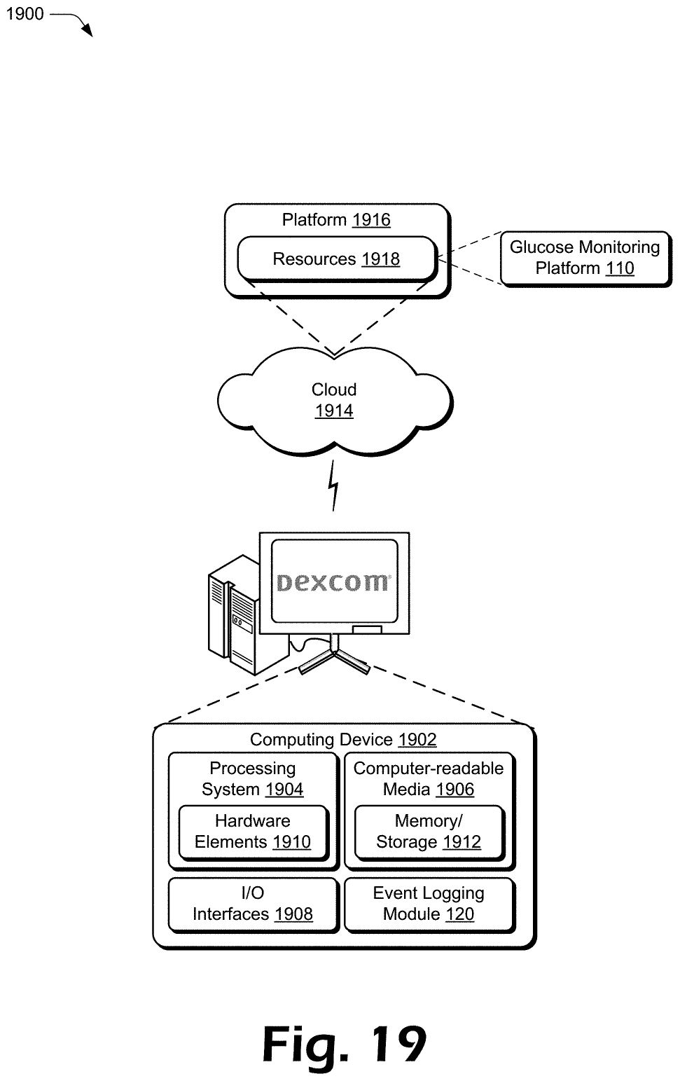

FIG. 19 illustrates an example of a system generally at 1900 that includes an example of a computing device 1902 that is representative of one or more computing systems and/or devices that may implement the various techniques described herein. This is illustrated through inclusion of the event logging module 120 and the glucose monitoring platform 110. The computing device 1902 may be, for example, a server of a service provider, a device associated with a client (e.g., a client device), an on-chip system, and/or any other suitable computing device or computing system.

The example computing device 1902 as illustrated includes a processing system 1904, one or more computer-readable media 1906, and one or more I/O interfaces 1908 that are communicatively coupled, one to another. Although not shown, the computing device 1902 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1904 is representative of functionality to perform one or more operations using hardware. Accordingly, the processing system 1904 is illustrated as including hardware elements 1910 that may be configured as processors, functional blocks, and so forth. This may include implementation in hardware as an application specific integrated circuit or other logic device formed using one or more semiconductors. The hardware elements 1910 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 1906 is illustrated as including memory/storage 1912. The memory/storage 1912 represents memory/storage capacity associated with one or more computer-readable media. The memory/storage component 1912 may include volatile media (such as random access memory (RAM)) and/or nonvolatile media (such as read only memory (ROM), Flash memory, optical disks, magnetic disks, and so forth). The memory/storage component 1912 may include fixed media (e.g., RAM, ROM, a fixed hard drive, and so on) as well as removable media (e.g., Flash memory, a removable hard drive, an optical disc, and so forth). The computer-readable media 1906 may be configured in a variety of other ways as further described below.

Input/output interface(s) 1908 are representative of functionality to allow a user to enter commands and information to computing device 1902, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1902 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 1902. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1902, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1910 and computer-readable media 1906 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1910. The computing device 1902 may be configured to implement particular instructions and/or functions corresponding to the software and/or hardware modules. Accordingly, implementation of a module that is executable by the computing device 1902 as software may be achieved at least partially in hardware, e.g., through use of computer-readable storage media and/or hardware elements 1910 of the processing system 1904. The instructions and/or functions may be executable/operable by one or more articles of manufacture (for example, one or more computing devices 1902 and/or processing systems 1904) to implement techniques, modules, and examples described herein.

The techniques described herein may be supported by various configurations of the computing device 1902 and are not limited to the specific examples of the techniques described herein. This functionality may also be implemented all or in part through use of a distributed system, such as over a “cloud” 1914 via a platform 1916 as described below.

The cloud 1914 includes and/or is representative of a platform 1916 for resources 1918. The platform 1916 abstracts underlying functionality of hardware (e.g., servers) and software resources of the cloud 1914. The resources 1918 may include applications and/or data that can be utilized while computer processing is executed on servers that are remote from the computing device 1902. Resources 1918 can also include services provided over the Internet and/or through a subscriber network, such as a cellular or Wi-Fi network.

The platform 1916 may abstract resources and functions to connect the computing device 1902 with other computing devices. The platform 1916 may also serve to abstract scaling of resources to provide a corresponding level of scale to encountered demand for the resources 1918 that are implemented via the platform 1916. Accordingly, in an interconnected device embodiment, implementation of functionality described herein may be distributed throughout the system 1900. For example, the functionality may be implemented in part on the computing device 1902 as well as via the platform 1916 that abstracts the functionality of the cloud 1914.

### CONCLUSION

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A method comprising:
receiving, at a computing device, a user input to log an event;
responsive to the user input, logging the event with a time of the event; and
displaying, in a user interface, an event representation of the event at a position on a glucose graph corresponding to the time of the event.

2. The method as described in claim 1, wherein the event comprises a meal consumed by the user.

3. The method as described in claim 2, wherein the user input comprises user input to capture an image of the meal consumed by the user using a camera of the computing device.

4. The method as described in claim 3, wherein the logging comprises storing the image of the meal with a capture time at which the image was captured using the camera.

5. The method as described in claim 3, wherein the displaying the event representation comprises displaying the image of the meal consumed by the user at the position on the glucose graph corresponding to a capture time at which the image was captured using the camera.

6. The method as described in claim 1, wherein the user input comprises at least one voice command to log the event.

7. The method as described in claim 1, wherein the user input comprises a selection of the event from a user interface that displays multiple events.

8. The method as described in claim 1, wherein the event comprises an activity performed by the user.

9. The method as described in claim 8, wherein the activity performed by the user comprises one of exercise, meditation, or sleep.

10. A method comprising:
obtaining, at a computing device, glucose measurements of a user, the glucose measurements collected by a glucose monitoring device;
displaying, by the computing device, a user interface that includes a glucose graph that plots the glucose measurements collected by the glucose monitoring device over a time period;
receiving meal data indicative of a meal consumed by the user and a time at which the meal was consumed; and
displaying, in the user interface, a meal representation for the meal consumed by the user, the meal representation overlaying the glucose graph at a position corresponding to the time at which the meal was consumed.

11. The method as described in claim 10, wherein the user interface further includes a selectable meal logging control, and wherein the receiving the meal data further comprises:
receiving, by the computing device, user input to select the meal logging control; and
capturing an image of the meal using a camera of the computing device.

12. The method as described in claim 11, further comprising determining the time at which the meal was consumed based on a time at which the image is captured using the camera of the computing device.

13. The method as described in claim 11, wherein displaying the meal representation comprises displaying the captured image of the meal as the meal representation for the meal consumed by the user.

14. The method as described in claim 10, wherein the meal data is received from a meal logging application.

15. The method as described in claim 10, wherein the meal representation is user selectable, and wherein the method further comprises:
receiving user input to select the meal representation; and
displaying additional information associated with the meal in the user interface.

16. The method as described in claim 15, wherein the additional information includes an additional glucose graph associated with at least one similar meal consumed by the user at a previous time.

17. The method as described in claim 15, wherein the meal representation comprises an image of the meal consumed by the user, and wherein the additional information comprises nutritional information obtained by processing the image of the meal consumed by the user to identify the meal consumed by the user.

18. The method as described in claim 10, wherein the meal representation includes a visual indication of a classification of the meal consumed by the user.

19. The method as described in claim 18, wherein the visual indication classifies the meal as being high in carbohydrates or low in carbohydrates.

20. The method as described in claim 18, wherein the visual indication classifies the meal as having a negative, neutral, or positive effect on the glucose measurements of the user.

21. The method as described in claim 10, further comprising:
receiving activity data indicative of an activity performed by the user and a time at which the activity was performed; and
displaying, in the user interface, an activity representation for the activity performed by the user, the activity representation overlaying the glucose graph at a position corresponding to the time at which the activity was performed.

22. The method as described in claim 21, wherein the activity data is received from a third-party application that is separate from a glucose application that displays the user interface.

23. The method as described in claim 10, wherein the glucose monitoring device comprises a wearable glucose monitoring device that is wirelessly coupled to the computing device.

24. A computing device comprising:
a display device;
one or more processors; and
a memory having stored thereon computer-readable instructions that are executable by the one or more processors to perform operations comprising:
obtaining, at the computing device, glucose measurements of a user, the glucose measurements collected by a glucose monitoring device;
displaying, on the display device, a user interface that includes a glucose graph that plots the glucose measurements collected by the glucose monitoring device over a time period;
receiving event data indicative of an event associated with the user and a time of the event; and
displaying, in the user interface, an event representation for the event, the event representation overlaying the glucose graph at a position corresponding to the time associated with the event.

25. The computing device as described in claim 24, wherein the event comprises a meal consumed by the user, and the event data comprises an image of the meal.

26. The computing device as described in claim 25, further comprising a camera, and wherein the image of the meal is captured by the camera.

27. The computing device as described in claim 24, wherein the event comprises an activity performed by the user.

28. The computing device as described in claim 27, wherein the activity performed by the user comprises exercise performed by the user.

29. The computing device as described in claim 28, wherein the event representation includes a visual indication of an intensity or duration of the exercise performed by the user.

30. The computing device as described in claim 24, wherein the event data is received from a third-party application.

31. The computing device as described in claim 24, wherein the event representation is selectable to view additional information associated with the event.

32. One or more computer-readable storage media having instructions stored thereon that are executable by one or more processors to perform operations comprising:
displaying a user interface that includes a glucose graph, the glucose graph plotting glucose measurements collected for a user by a glucose monitoring device over a time period; and
displaying, in the user interface, an event representation for an event associated with the user, the event representation overlaying the glucose graph at a position corresponding to a time of the event.

33. An apparatus comprising:
a receiving means for receiving a user input to log an event;
a logging means for logging the event with a time of the event responsive to the user input; and
a displaying means for displaying, in a user interface, an event representation of the event at a position on a glucose graph corresponding to the time of the event.
